(19) United States
(12) Patent Application Publication
(10) Pub. No.: US 2007/0178521 A1
(43) Pub. Date: Aug. 2, 2007

(54) Assay chip

(76) Inventors: Yoshiki Sakaino, Asaka-shi (JP); Yukio Sudo, Asaka-shi (JP); Yoshihiko Abe, Asaka-shi (JP); Bo Yang, Asaka-shi (JP)

Correspondence Address: BIRCH STEWART KOLASCH & BIRCH, PO BOX 747, FALLS CHURCH, VA, 22040-0747, US

(21) Appl. No.: 10/592,748

(22) PCT Filed: Mar. 15, 2005
(86) PCT No.: PCT/JP05/05062

§ 371 Date: Sep. 14, 2006

(30) Foreign Application Priority Data
Mar. 16, 2004 (JP) 2004-073965
Jul. 23, 2004 (JP) 2004-215302

(51) Int. Cl. G01N 33/53; C12M 3/00

(52) U.S. Cl. 435/7.1; 435/287.2; 977/902

(57) ABSTRACT

An integrated assay chip for assaying one or more components of a specimen, which comprises: (1) one or more pretreatment elements for pretreating a specimen; (2) one or more multilayer dry assay elements capable of assaying one or more components of the pretreated specimen; and (3) one or more flow channels connecting the pretreatment element and the multilayer dry assay element is provided.

### TECHNICAL FIELD

The present invention relates to an integrated assay chip and a method for assaying one or more components contained in a specimen such as blood, urine, body fluid, tissue, cell, food, waste fluid, bond water, river water, seawater or rainwater.

### BACKGROUND ART

Hitherto, methods for diagnosing human diseases using blood, urine, or the like as a specimen have been performed for a long time as conveniently diagnosable methods without damaging the human body.

As one of the methods, there is a wet chemistry assay method. This method is a method using a so-called solution reagent and has a long history. Reagents for detection have been developed for a large number of test items and there are various measuring equipments including from handy small ones to large fully automatic ones. A specimen to be used in the wet chemistry is plasma, serum, urine, or the like and usually, whole blood itself is not used as the specimen.

In the wet chemistry, the reagents may be divided into several groups in consideration of their stability during storage and then mixed at dissolution and preparation thereof or it is possible to divide the procedure of adding the reagents into several steps.

Furthermore, since appropriate amounts of reagents can be dissolved and prepared depending on the number of analytes to be measured, a reagent cost per measurement can be reduced. Although it is complex and tedious to combine and automate handling of many solutions, development of clinical laboratory equipments is historical and socially highly desired and hence efficient automatic equipments have been already developed and put into practical use even in the fields where any of small, medium, and large treating capacities are necessary. However, the wet chemistry assay method could not often satisfy the requirements by doctors in private practice and by emergency hospitals that are required to immediately provide their diagnosis results.

On the other hand, recently, home care has been proposed as a measure to correspond medical cost inflation based on the backgrounds of rapid change to aging society, development of advanced medical treatment, and the like. The home care is considered to be one core of medical service system in future and specific methods for performing the care have been investigated.

A fundamental point of home care is that a patient is under constant appropriate observation, guidance, and control of a doctor while dwelling in the comfort of patient's home and can receive an appropriate medical service as the need arises. For example, in the cases of chronic patients and aged persons, they are in stable physiological conditions unless no rapid change in clinical conditions occurs and hence it is most important to continue a constant medical treatment and continuous monitoring of the therapeutic effect.

Although such continuous monitoring is easy for patients in hospital, the monitoring is practically extremely difficult in the cases where patients are distributed in respective homes and available information is usually only subjective ones, e.g., monitor records of symptoms by nursing persons and complaint of the patients themselves except measured values of body temperature and body weight.

When information of blood test is continuously obtained, routine control by a doctor becomes easy and the treatment becomes more rapid and appropriate, so that benefit thereof is immeasurable. Moreover, the patient may have a deep sense of security that routine conditions of the disease are reported to and checked by the doctor and hence it is easily understood that it may provide psychological support until recovery.

Furthermore, when a blood test of the patient can be carried out at home without visiting the patient by a doctor or a nurse according to their direction, more rapid and more appropriate treatment becomes possible. It may benefit all the homes in view of the time required for house visit but, in particular, it may provide a large advance and benefit for medical treatment in difficultly accessible locations, distant places, isolated islands, and undeveloped places.

In addition, for example, in the case of diabetic outpatients, when they can measure blood sugar levels several times in a daily life and bring the results at hospital visit, a doctor responsible to their treatment can more accurately comprehend the conditions of the patients. As a blood level measuring apparatus for diabetic patients, an apparatus capable of semi-quantitative determination has been developed and some kinds of the apparatus can be used at home but the apparatus is low in accuracy, so that it is not applicable to the above purpose.

It need scarcely be said that a blood assay method is desired, which is carried out (1) with a small size apparatus, (2) conveniently, (3) using a minute amount of blood, (4) with regard to many items, (5) rapidly, and (6) accurately. In particular, in the case that aged persons (optionally children) are targeted, it is desired to develop an assay method which necessitates a minute amount of the blood.

For the purpose, there is proposed an integrated analyzer for home use using a wet chemistry assay method (Patent Reference 1), which discloses a small-sized health-care device that comprises a blood-collecting unit, a filtration unit for obtaining plasma from the blood through filtration, a separation unit of separating the blood to obtain serum, and units for determining the pH value, the oxygen concentration, the carbon dioxide concentration, the sodium concentration, the potassium concentration, the calcium concentration, the glucose concentration and the lactic acid concentration in the blood component, in which these units are compactly integrated. But the apparatus is not satisfactory in view of easiness and simplicity as well as size.

On the other hand, a so-called dry chemistry assay method has been developed, wherein reagents necessary for detection of specific components are contained in a dry state.

In the dry chemistry, all the reagents necessary for qualitative and quantitative assay are incorporated in an assay element (dry assay element) such as a reagent paper, a disposable electrode, or a magnetic material. Basically, it is a disposable type capable of measuring one item per a specimen and it is possible to carry out blood assay conveniently and rapidly with a relatively minute amount (about 10 μl) of blood. A large number of analyzers using the dry chemistry assay method (dry chemistry assay apparatus) have been developed and commercialized, and FUJI DRI-CHEM (manufactured by Fiji Photo Film Co., Ltd.), Ecta Chem (manufactured by Eastman Kodak Co., U.S.A.), Dry Lab (manufactured by Konica Corporation), Spot Chem (manufactured by Kyoto Dai-ichi Kagaku K.K.), Reflotron (manufactured by Boehringer Manheim, Germany), Seralyzer (manufactured by Miles Laboratory, U.S.A.), and the like have been commercially available.

To that effect, the dry chemistry assay method is better than the conventional wet chemistry assay method from the viewpoint that it is small-sized and enables rapid and simplified assay of components of a specimen, and has succeeded in bringing about some results, but it still has some problems.

For example, when the specimen is whole blood and the component to be tested is in serum, for example, lactate dehydrogenase, then the multilayer dry assay element for assaying lactate dehydrogenase is described in Patent References 2 and 3. In this case, however, the specimen is not directly applied to the multilayer dry assay element but must be indispensably pretreated (for removing the blood cell component from whole blood).

In such case, when the specimen is blood, whole blood is usually not used, and, after removal of blood cells, measurement is frequently conducted in the form of plasma or serum using the dry chemistry assay method. As methods for removing blood cells, there is a method using centrifugal force, filter, or the like, and a large number of blood cell separators have been developed. As blood cell separators operable with a minute amount of blood, those described in Patent References 4 and 5 are proposed, for example.

The necessity of such a blood cell separator adds a step of operating the device, invites decrease of easiness and simplicity, and furthermore induces an undesirable situation of increasing a necessary blood amount.

In the dry chemistry analyzer, by combining a centrifugal separator and a multilayer film for assay, which forms a film of reagent used in the dry chemistry assay method as a dry assay element (hereinafter, including this embodiment, referred to as “a multilayer dry assay element”), a dry chemistry analyzer necessitating no blood cell-separating operation and capable of measuring many items at the same time can be manufactured. For example, Patent References 6 and 7 discloses an assay cartridge, in which a multilayer dry assay element and a flow channel are integrated. A blood cell separation is conducted in the flow channel by an external force due to a combination of the cartridge and a centrifugal separator. However, in these methods, the necessary amount of blood cannot be sufficiently decreased and the methods are not satisfactory in view of rapidness.

On the other hand, when the specimen is whole blood and the component to be tested exists in blood cells, for example, hemoglobin A1C, then the blood cells in the specimen must be disrupted, different from those in the case where the serum component is assayed. The multilayer dry assay element for assaying hemoglobin A1C is described in Patent Reference 8. In this case, however, the specimen is not directly applied to the dry assay element but must be indispensably pretreated (operation for disrupting the blood cells in the specimen).

For example, in an immunoassay based on antigen-antibody reaction, the specimen (essentially plasma) must be often diluted for the purpose of rapidly promoting the antigen-antibody reaction. For example, the multilayer dry immunoassay element is described in Patent References 9 to 11. In this case, however, the specimen is not directly applied to the multilayer dry immunoassay element but must be previously diluted in many cases.

For example, when the analyte in a specimen is a high-molecular-weight substance such as saccharified protein (e.g., glycoalbumin, hemoglobin A1C), then the specimen may be degraded with a protease and then the component in the degraded product may be assayed.

As mentioned hereinabove, specimens require some pretreatment in many cases before applied to dry assay elements. The pretreatment complicates the measurement operation and lowers the measurement accuracy, and in addition, it often brings about some unfavorable conditions in that the possibility that assayers may be contaminated by specimens increases.

On the other hand, recently, a large number of micro-fabricated chips have been proposed, which utilizes a micro-fabrication technique. For example, Patent Reference 12 discloses a system for effecting liquid transfer utilizing centripetal force generated by rotation of a platform. However, in a micro-fluid system, there is a major defect that it is difficult to mix an analyte and a reagent for detection since a fluid to be transferred forms a laminar flow and hence the system is problematic as an assay method for use in home care.

[Patent Reference 1] JP-A-2001-258868

[Patent Reference 2] JP-A-62-093662

[Patent Reference 3] JP-A-62-228947

[Patent Reference 4] JP-A-9-196911

[Patent Reference 5] JP-A-11-38001

[Patent Reference 6] JP-T-2001-512826

[Patent Reference 7] JP-T-2002-514755

[Patent Reference 8] JP-A-9-166594

[Patent Reference 9] JP-A-1-237455

[Patent Reference 10] JP-A-1-321360

[Patent Reference 11] JP-A-1-321361

[Patent Reference 12] JP-A-2003-28883

### DISCLOSURE OF THE INVENTION

An object of the invention is to provide an assay chip capable of giving high-accuracy assay data in a rapid and simplified manner in the field where specimens are collected.

Another object of the invention is to provide an assay chip which does not require any substantial pretreatment of specimen collected by assayer and with which the assayer enables safe assay with little possibility of contamination with specimen.

Still another object of the invention is to provide an assay chip (e.g., a blood assay chip) capable of use for home care, i.e., an assay chip capable of measuring many kinds of assay items rapidly and conveniently from a minute amount of whole blood with miniaturizing a measuring apparatus without decreasing accuracy.

As a result of extensive studies for solving the above problems, the present inventors have found that, when a specimen pretreatment element (e.g., blood cell separation element, hemolysis element, dilution element) is bonded to and integrated with a multilayer dry assay element either directly or via a flow channel therebetween, then an assay chip that solves the above problems can be provided, and on the basis of this finding, we have completed the invention.

1. An integrated assay chip for assaying one or more components of a specimen, comprising: (1) one or more pretreatment elements for pretreating a specimen; (2) one or more multilayer dry assay elements capable of assaying one or more components of the pretreated specimen; and (3) one or more flow channels connecting the one or more pretreatment elements and the one or more multilayer dry assay elements.

2. An integrated assay chip for assaying one or more components of a specimen, comprising: (1) one or more pretreatment elements for pretreating a specimen; and (2) one or more multilayer dry assay elements capable of assaying one or more components of the pretreated specimen, wherein the one or more pretreatment elements and the one or more multilayer dry assay elements are connected inside the integrated assay chip.

3. The integrated assay chip according to the item 1 or 2, wherein the pretreatment is at least one selecting from the group consisting of a blood cell separation, hemolysis, dilution of specimen, degradation of protein, denaturation of protein, removal of endogenous substance and antigen-antibody reaction.

4. The integrated assay chip according to the item 1 or 2, wherein the one or more pretreatment elements (1) include an element for a blood cell separation, which contains at least one of a porous material and a water-insoluble substance that has an equivalent circle diameter of not more than 5 μm and a length equal to or longer than an equivalent circle diameter.

5. The integrated assay chip according to the item 1 or 2, wherein the one or more pretreatment elements (1) include an element for a blood cell separation, which contains any one of a glass fiber and a glass fiber filter paper.

6. The integrated assay chip according to the item 1 or 2, wherein the one or more pretreatment elements (1) include an element for a blood cell separation, which includes a blood filtration unit containing a glass fiber filter paper and a microporous membrane.

7. The integrated assay chip according to any one of the items 4 to 6, wherein the element for a blood cell separation is an element utilizing a centrifugal force.

8. The integrated assay chip according to any one of the items 1 and 3 to 7, wherein the one or more flow channels includes a micro flow channel having an equivalent diameter of 3 mm or less.

9. The integrated assay chip according to any one of the items 1 to 8, which comprises a cartridge, in which the cartridge contains three elements of the one or more pretreatment elements, the one or more multilayer dry assay elements and the one or more flow channels, or two elements of the one or more pretreatment elements and the one or more multilayer dry assay elements.

10. The integrated assay chip according to any one of the items 1 to 8, wherein at least one of the one or more pretreatment elements and the one or more flow channels is formed on a substrate or inside thereof by a micro-fabrication technique, and the one or more multilayer dry assay elements are bonded to the one or more flow channel.

11. A specimen assay method, comprising: applying a specimen to the integrated assay chip of any one of the items 1 to 10; and leading the specimen through the one or more pretreatment elements and the one or more multilayer dry assay elements in this order.

### BREIF DESCRIPTION OF THE DRAWINGS

FIG. 1 shows a flow channel pattern in Example 1.

FIG. 2 shows an integrated assay chip of Example 1.

FIG. 3 shows a calibration curve prepared for measurement of HbA1C by the use of an integrated assay chip of Example 1.

FIG. 4 is a schematic diagram showing a layout of an element for introducing whole blood, a blood cell separation element, a multilayer dry assay element and a flow channel in the assay chip of the present invention.

FIG. 5 shows a sectional view of one embodiment in the assay chip of the present invention.

FIG. 6 shows a schematic view of one example of layout of optical system in case of using the assay chip for measurement

FIG. 7 shows a schematic view illustrating one embodiment in the assay chip of Example 2.

FIG. 8 shows a photograph illustrating one embodiment in the assay chip of Example 2.

FIG. 9 shows a photograph after introduction of whole blood in one embodiment in the assay chip of Example 2.

FIG. 10 shows a photograph illustrating that a color-developing reactive reagant initiate coloring by suction with a Termo-syringe after introduction of whole blood in one embodiment in the assay chip of Example 2.

### DESCRIPTION OF THE REFERENCE NUMERALS SIGNS

1 Element for introducing whole blood
2 Blood cell separation element (Pretreatment element) (1)
3 One or more multilayer dry assay elements (2) capable of assaying one or more components of plasma
4 One or more flow channels (3) connecting the above (a) and (b)
100 Measuring apparatus
11 Assay chip setting portion
12 Light source
13 Light variant portion (Neutral density filter)
14 Wavelength tunable portion (Interference filter)
15a, 15b, 15c Lenses
16 Area sensor (CCD)
17 Computer (Image-processing apparatus)
31 Inlet for introducing a specimen
32 Hemolytic reagent
33 Multilayer dry assay element
34 Flow channel
35 Air extractor
40 Assay chip
41 Pipe (Inlet for introducing whole blood)
42a Glass fiber filter paper
42b Polysulfone porous membrane
43 Multilayer dry assay element
44 Flow channel
45 Pipe
46 Upper member
47 Lower member

### BEST MODE FOR CARRYING OUT THE INVENTION

Embodiments of the invention are described in detail hereinunder.

[Integrated (One-Piece) Assay Chip]

The invention relates to an integrated assay chip for assaying one or more ponents in a specimen.

As a first embodiment thereof, the assay chip of the invention comprises at least (1) one or more pretreatment elements for pretreating a specimen, (2) one or more multilayer dry assay elements capable of assaying one or more components of the pretreated specimen, and (3) one or more flow channels connecting the above pretreatment element and the above multilayer dry assay element.

As a second embodiment thereof, the assay chip of the invention comprises at least (1) one or more pretreatment elements for pretreating a specimen, and (2) one or more multilayer dry assay elements capable of assaying one or more components of the pretreated specimen, wherein the above pretreatment element and the above multilayer dry assay element are connected inside the assay chip.

As another embodiment thereof for assaying components of whole blood, the assay chip of the invention may have a detachable blood-collecting element, in addition to (1) one or more pretreatment elements for pretreating a specimen, (2) one or more multilayer dry assay elements capable of assaying one or more components of the pretreated specimen, and (3) one or more flow channels connecting the above pretreatment element and the above multilayer dry assay element.

As still another embodiment thereof, the assay chip of the invention may include an element for introducing a specimen thereinto (drop), an element for determining the amount of the specimen, an element for determining the amount of the specimen to be led into the multilayer dry assay element, and an element for mixing a specimen and a reaction solution, in addition to (1) one or more pretreatment elements for pretreating a specimen, (2) one or more multilayer dry assay elements capable of assaying one or more components of the pretreated specimen, and (3) one or more flow channels connecting the above pretreatment element and the above multilayer dry assay element.

In the invention, “integrated (one-piece)” means that measurement of all items to be measured on the components of the specimen introduced into the above assay chip can be completed without discharging the components from the assay chip.

As the integrated assay chip, the following form (i) or (ii) may be mentioned.

(i) A form wherein the above three elements (1) to (3) (or the two elements (1) and (2)) are integrated by incorporating them into one cartridge.

(ii) A form wherein either of the above (1) or (3) or both of them are made on or in a substrate board using a so-called micro-fabricating technique, and if desired, the multilayer dry assay element (2) is bonded to the flow channel (3).

In the above (i), as the material for constituting the cartridge, resins such as rubbers and plastics, and silicon-containing substances may be mentioned.

Examples of the resins include polymethyl methacrylate (PMMA), polycyclic olfeins (PCO), polycarbonate (PC), polystyrene (PS), polyethylene (PE), polyethylene terephthalate (PET), polypropylene (PP), polydimethylsiloxane (PDMS), natural rubber, synthetic rubbers, and derivatives thereof

As the silicon-containing substances, glass, quartz, amorphous silicon such as silicon wafer, silicones such as polymethylsiloxane may be mentioned.

Of these, preferred are PMMA, PCO, PS, PC, glass, and silicone wafer. These materials are transparent, and are more preferable in case of using a photometry as mentioned below. In this case, it is not necessary that all of materials constituting the cartridge are transparent, and it is necessary to be able to look the multilayer dry assay element subjected to the photometry outside the cartridge. In the cartridge, a window frame may be provided for looking the multilayer dry assay element, and only a portion inside the window frame may be made of the transparent material.

The shape and size of the cartridge may be any shape and size as far as the shape and size fall within the range that is easy to handle. Specifically, for example, those having a rectangular basal plane whose one side is about 10 to 50 mm and having a thickness of about 2 to 10 mm may be mentioned as preferred examples of the shape and size.

The pretreatment element (1) and the flow channel (3) have the constitution as mentioned below, and the micro-fabricating technique used in the above (ii) may be applied to a preparation of the components (1) and (3).

In the above (ii), the above (1) and/or (3) can be manufactured on a substrate by a micro-fabrication technique. Examples of the material to be used in the substrate include metals, silicon, tetrafluoroethylene, glass, ceramics, plastics, and rubbers.

Examples of the plastics include PCO, PS, PC, PMMA, PE, PET, PP, and the like. Examples of the rubbers include natural rubber, synthetic rubbers, silicone rubbers, PDMS, and the like.

As the silicon-containing substances, glass, quartz, amorphous silicon such as silicone wafer, silicones such as polymethylsiloxane may be mentioned.

Especially preferred examples include PMMA, PCO, PS, PC, PET, PDMS, glass, silicone wafer, and the like. These materials are transparent, and are more preferable in case of using a photometry as mentioned below. In this case, it is not necessary that all portions of the substrate are transparent, and it is necessary to be able to look the multilayer dry assay element outside the assay chip. In the assay chip, a window frame may be provided for looking the multilayer dry assay element, and only a portion inside the window frame may be made of the transparent material.

The shape and size of the substrate may be any shape and size as far as the shape and size fall within the range that is easy to handle. Similar to the shape and size of the cartridge in (i), for example, those having a rectangular basal plane whose one side is about 10 to 50 mm and having a thickness of about 2 to 10 mm may be mentioned as preferred examples of the shape and size.

Particularly, in case of making the flow channel (3) using the micro-fabricating technique, examples of the material usable for solid substrates in the invention are metal, silicon, Teflon (registered trade mark), glass, ceramics and plastics. Above all, preferred are metal, silicon, Teflon (registered trade mark), glass and ceramics from the viewpoint of the heat resistance, pressure resistance, solvent resistance and light transparency thereof. More preferred is glass.

Examples of the micro-fabrication technique for manufacturing (1) and/or (3) include methods described in Microreactor—Shin Jidai no Gosei Gijutsu—(supervising editor: Professor Jun-ichi Yoshida, Kyoto University Graduate School, Department of Technology, published by CMC, 2003), Bisai Kako Gijutsu, Article of Application —Application to Photonics, Electronics, and Mechatronics—(edited by Event Committee of Society of Polymer Science, Japan, published by NTS, 2003), and so forth.

Representative methods include LIG technique using X-ray lithography, high aspect ratio photolithography using EPON SU-8, micro-electric discharge machining (μ-EDM), high aspect ratio processing of silicon by deep RIE, hot emboss processing, laser beam lithography, laser processing, ion beam processing, and mechanical micro-cutting using a micro-tool made of a hard material such as diamond, and the like. These technologies may be used solely or in combination. Preferred micro-processing technologies are LIGA technique using X-ray lithography, high aspect ratio photolithography using EPON SU-8, micro-electric discharge machining (μ-EDM), and mechanical micro-cutting.

These micro-fabrication techniques can be also applied to the manufacture of (1) and/or (3) in the above (i).

The above (1) and/or (3) in the invention can be also manufactured by pouring and solidifying a resin in a pattern, as a mold, formed on a silicon wafer using a photoresist (molding process). For the molding process, a silicone resin including PDMS or a derivative thereof as a representative can be employed.

A junction technique can be used at the time when the integrated assay chip of the invention is assembled. Usual junction technique is classified into solid-phase junction and liquid-phase junction. The representative junction methods generally used are pressure welding and diffusion junction as the solid-phase junction, and welding, eutectic bonding, soldering, and adhesion as the liquid-phase junction.

Furthermore, at the assembly, it is desired to use a highly precise junction method capable of maintaining dimensional accuracy without destruction of the microstructures such as flow channels which may occur owing to deterioration or severe deformation of the material by high-temperature heating. As such techniques, there may be mentioned silicon direct junction, anodic juncture, surface-activated junction, direct junction using hydrogen bond, junction using an aqueous HF solution, Au—Si eutectic bonding, void-free adhesion, and the like.

Moreover, junction using ultrasonic wave, laser, or the like and junction using an adhesive, an adhesive tape, or the like may be employed and also junction may be achieved merely by applying pressure.

In the case that the flow channel and the multilayer dry assay element are combined, a large number of common methods may be applicable, for example, an adhesive, an adhesive double coated tape, welding with ultrasonic wave, use of a photocuring agent, use of a surface-treating agent, and the like. Moreover, depending on each constitutive material and form, there is a case that mere continuous pressurization is sufficient. In any case, any method is suitable as far as it is a method which achieves no leakage of a specimen such as plasma.

[Pretreatment Element]

The following will describe (1) pretreatment element among the above three elements of (1) to (3).

The pretreatment in the invention indicates all or part of the treatments necessary before subjecting a sampled specimen to the dry assay element. The pretreatment element means an element which conducts the pretreatment of the specimen. Examples of the pretreatment include blood cell separation, hemolysis, dilution of the specimen, decomposition of a protein, denaturation of a protein, removal of an endogenous substance, antigen-antibody reaction and the like.

The pretreatment element may be a single pretreatment element capable of conducting these plurality of pretreatments or may be plurality of pretreatment elements wherein each element conducts a pretreatment different from each other. Moreover, these elements may be connected through the flow channel or may be incorporated into the flow channel.

The pretreatment element usually comprises a member composing the pretreatment element and reagent(s) for conducting the pretreatment as occasion demands. The member composing the pretreatment element may be contained in the aforementioned cartridge or may be built on or in the substrate.

The pretreatment element itself may be in a flow channel shape. From the viewpoint of rapid progress of the reaction, the element may be a porous substance. The porous substance may be arranged in the pretreatment element (or an element composing the pretreatment element) in a flow channel shape or in the pretreatment element in a shape other than the flow channel shape, or the flow channel itself may be a porous substance. Examples of the porous substance herein include a filter paper, a membrane, a glass fiber, a glass fiber filter, a fiber, a non-woven fabric, and combinations thereof.

Moreover, the pretreatment element may be fine particles such as beads. Examples of the fine particles include glass beads, silicon beads, polymer beads, latex beads, nanoparticles, magnetic particles, amorphous silicon beads, and combinations thereof. As examples of such beads, the technologies described in JP-A-2004-61496 and JP-A-5-87812 can be employed.

Furthermore, the pretreatment element can be prepared on a solid substrate by the micro-fabrication technology as mentioned above. In this case, the pretreatment element may be formed as substantially porous one by the micro-fabrication technology as mentioned above. In this case, the pretreatment element is preferably in a pillar shape.

In the case that the pretreatment element contains reagent(s), it is desirable to retain the reagent(s) for pretreatment in the pretreatment element. As the method for introducing the reagent(s) into the pretreatment element, a spotting method, a screen printing method, a nanocontact printing method, an ink-jet method, or the like can be suitably utilized depending on the constitution of the pretreatment element. Moreover, after the reagent(s) is applied on a base made of polyethylene terephthalate, a cellulose acetate derivative, or the like beforehand, the resulting article may be adhered to the pretreatment element. Furthermore, after the reagent(s) is contained in (or adsorbed on, fixed to, dispersed in) the porous substance as mentioned above, the resulting article may be introduced into the pretreatment element.

{Blood Cell Separation}

As one specific example of the pretreatment in the invention, blood cell separation may be mentioned. The blood cell separation in the invention means a step of separating blood cells from whole blood and isolating plasma or serum.

In the invention, the form of the blood cell separation element may be any form as far as the element can separate blood cells from whole blood to afford plasma or serum, and it is possible to have any form, e.g., a linear form, a curved form, and the like.

Moreover, in the invention, any element can be utilized as the blood cell separation element, which is used in hitherto known blood cell separation. For example, an element utilizing centrifugal force, an element using filtration, and the like may be mentioned. Additionally, they may be used in combination.

In the case of the element utilizing centrifugal force, whole blood is injected to an assay chip, blood cells are separated by rotating the chip on a centrifugal separator, and the resulting plasma can be introduced into the multilayer dry assay element directly or through the flow channel. As the element utilizing centrifugal force, any form may be applicable as far as it has a form capable of utilizing the centrifugal separator and it can separate blood cells and introduce the resulting plasma into the multilayer dry assay element through the flow channel. For example, a form having a concave portion where solid components including blood cells are to be placed after blood cell separation can be mentioned as a preferred specific example.

In the invention, it is preferable to have a filtration part as the blood cell separation element. As a filter material for use in the filtration part, any filter material which is hitherto known may be utilized but a porous substance is preferable. The porous substance includes a filter paper, a membrane, a glass fiber, a glass fiber filter, and the like. Moreover, they may be used in combination. Furthermore, methods described in JP-A-11-6829, JP-A-11-38001, JP-A-11-38002, JP-A-11-38003, and JP-A-11-237378 can be employed, for example.

Additionally, the filtration part may be, for example, in a micro-pillar form which is formed by utilizing the above micro-fabrication technology.

Moreover, the filtration part may be a water-insoluble substance that has an equivalent circle diameter of not more than 5 μm and a length equal to or longer than an equivalent circle diameter.

The water-insoluble substance includes silicon, glass, polystyrene (PS), polyethylene terephthalate (PET), polycarbonate (PC), a polyimide known as a trademark of Kevlar or the like, a glass fiber, a glass fiber filter paper, a polyethylene terephthalate (PET) fiber, a polyimide fiber, and the like.

The water-insoluble substance is not necessarily limited to a fiber as far as the substance is a water-insoluble substance that has an equivalent circle diameter of not more than 5 μm and a length equal to or longer than an equivalent circle diameter. For example, there may be placed and used one molded into a shape in a pillar form generally called micro-pillar or nano-pillar using a micro-fabrication technology or a processing technology such as μTAS. Various methods have been known as methods for preparing the micro-pillar or nano-pillar but use may be made of a method of leaving silicon in a pillar form by exposing a silicon wafer to a light and etching the wafer or an imprinting method wherein columnar protrusions are formed by attaching a dented mold to a resin by pressure and then peeling it.

Furthermore, the water-insoluble substance is not necessarily limited to the shape in a pillar form. It is sufficient to prepare a structure having an equivalent circle diameter of not more than 5 μm and a length equal to or longer than an equivalent circle diameter by a photolithography using a photocuring resin or the like. In this case, a mechanical strength is imparted by preparing a structure wherein further crosslinking is introduced between the structures, whereby a structure satisfying both of filtration performance and mechanical strength can be manufactured. The shapes of the structure include structure wherein crosslinking is introduced between pillars, structure wherein crosslinking is introduced between fibers, mesh structure in a double cross, checked, or honeycomb form and crosslinked structure thereof, and the like.

Also, the blood cell separation element may include a blood filtration unit having a glass fiber filter and a microporous membrane therein. The blood filtration unit is especially preferred since it enables efficient separation of plasma and serum from a blood specimen even though the specimen is a minor amount of blood.

The following will describe the blood filtration unit.

The glass fiber filter paper has a density of preferably about 0.02 to 0.3, more preferably about 0.02 to 0.2, particularly preferably about 0.02 to 0.15 and a retained particle size of preferably about 0.8 to 9 μm, particularly preferably about 1 to 5 μm. The filtration can be more rapidly and smoothly effected by treating the surface of glass fiber with a hydrophilic polymer by the method as described in JP-A-2-208565 or JP-A-4-208856. Moreover, lectin, another reactive reagent, or a modifier may be incorporated into a glass fiber filter paper or the surface of glass fiber can be treated with lectin. The glass fiber filter paper can be used without any treatment. A laminate of two or more sheets of the glass fiber filter papers can be also used.

Moreover, if necessary, the filter paper can be prepared and laminated by combining glass fibers having different density and other properties.

The microporous membrane does not hemolyze to such an extent that the analyzed values are substantially affected, and can specifically separate a blood cell and plasma from a whole blood.

The microporous membrane has a pore size of preferably smaller than the retained particle size of glass fiber filter paper and 0.2 μm or more, more preferably 0.3 to 8 μm, further preferably 0.5 to 4.5 μm, particularly preferably 0.5 to 3 μm.

Moreover, porosity thereof is preferably high and, specifically, the porosity is in the range of preferably about 40% to about 95%, more preferably about 50% to about 95%, further preferably about 70% to about 95%.

Examples of the microporous membrane include polysulfone membranes, fluorine-containing polymer membranes, cellulose acetate membranes, nitrocellulose membranes, and the like. Moreover, those whose surface is subjected to hydrophilic treatment by hydrolysis or with a hydrophilic polymer, an activator, or the like can be also employed.

As microporous membranes of fluorine-containing polymers, there may be mentioned microporous matrix membranes comprising polytetrafluoroethylene fibril (microfiber) described in JP-T-63-501594 (pamphlet of International Publication No. 87/02267), Gore-Tex (manufactured by W. L. Gore and Associates), Zitex (manufactured by Norton), Poreflon (manufactured by Sumitomo Electric Industries, Ltd.), and the like. In addition, use can be also made of microporous membranes of polytetrafluoroethylene described in Examples 3 and 4 of U.S. Pat. No. 3,268,872, Examples 3 and 4 of U.S. Pat. No. 3,260,413, JP-A-53-92195 (U.S. Pat. No. 4,201,548), and so forth, microporous membranes of polyvinylidene fluoride described in U.S. Pat. No. 3,649,505, and the like.

As the structure, use may be made of any of unoriented ones, uniaxially oriented ones, biaxially oriented ones, non-laminated one layer types, laminated two layer types, membranes laminated onto other membrane structures such as fibers, and the like. As the structure, use may be also made of a fibril structure.

A non-laminated type microporous membrane which has a fibril structure or is uniaxially oriented or biaxially oriented can be converted into a microporous membrane having a large porosity and a short filtration length by orientation. The microporous membrane having a short filtration length is preferred in view of high accuracy in quantitative determination because clogging induced by tangible components (mainly erythrocytes) in blood hardly occurs and the time required for separation of blood cells from plasma is short.

At preparation of these microporous membranes of fluorine-containing polymers, one or two or more fluorine-containing polymers may be mixed or they may be mixed with one or two or more polymers or fibers containing no fluorine, followed by film formation.

The microporous membrane of the fluorine-containing polymer can be subjected to a physical activation treatment (preferably glow discharge treatment or corona discharge treatment) described in JP-A-57-66359 (U.S. Pat. No. 4,783,315) onto at least one surface of the microporous membrane to render the surface of the microporous membrane hydrophilic, whereby adhesiveness of an adhesive to be used for partial adhesion to an adjacent microporous membrane can be strengthened.

The microporous membrane of the fluorine-containing polymer has a low surface tension as it is and hence even when it is intended to use the membrane as a filter material, an aqueous liquid specimen is repelled and is difficult to disperse and permeate into the surface and inside of the membrane. The problem that an aqueous liquid specimen is repelled can be solved by impregnating the microporous membrane of the fluorine-containing polymer with a surfactant in an amount sufficient to substantially impart hydrophilicity to the outer surface and the surface of inner voids of the microporous membrane of the fluorine-containing polymer, as a nieans for imparting hydrophilicity to the microporous membrane of the fluorine-containing polymer to enhance hydrophilicity thereof.

In order to impart hydrophilicity to the microporous membrane of the fluorine-containing polymer to an extent sufficient not to repel an aqueous liquid specimen and to diffuse, permeate, and transfer it into the surface and inside of the membrane, the surface of voids of the microporous membrane is covered with a surfactant in an amount of preferably about 0.01% to about 10%, more preferably about 0.1% to about 5.0%, further preferably 0.1% to 1% of void volume of the microporous membrane of the fluorine-containing polymer. For example, in the case of the microporous membrane of the fluorine-containing polymer having a thickness of 50 μm, the amount of the surfactant to be used for impregnation is, in general, preferably in the range of 0.05 g/m² to 2.5 g/m². As methods for impregnating the microporous membrane of the fluorine-containing polymer with a surfactant, there may be mentioned a method of immersing the microporous membrane of the fluorine-containing polymer in a solution of the surfactant in an organic solvent, e.g., an alcohol, an ester, or a ketone, having a low boiling temperature, preferably a boiling temperature ranging from about 50° C. to about 120° C., to permeate the solution substantially sufficiently into the inner voids of the microporous membrane, subsequently lifting up the microporous membrane from the solution, and drying the membrane by blowing air, preferably warm air.

As the surfactant for use in the hydrophilic treatment of the microporous membrane of the fluorine-containing polymer, any of nonionic, anionic, cationic, and amphoteric surfactants can be used.

Of these surfactants, nonionic surfactants are preferred owing to relatively low action to hemolyze erythrocytes. Examples of the nonionic surfactant include alkylphenoxypolyethoxy ethanols, alkylpolyether alcohols, polyethylene glycol monoesters, polyethylene glycol diesters, higher alcohol ethylene oxide adducts (condensates), polyhydric alcohol ester ethylene oxide adducts (condensates), higher fatty acid alkanolamides, and the like.

Specific examples of the nonionic surfactant include the following. As the alkylphenoxypolyethoxy ethanols, there may be mentioned isooctylphenoxypolyethoxy ethanol:
(Triton X-100: containing 9 to 10 oxyethylene units on average),
(Triton X-45: containing 5 oxyethylene units on average),
nonylphenoxypolyethoxy ethanol:
(IGEPAL CO-630: containing 9 oxyethylene units on average),
(IGEPAL CO-710: containing 10 to 11 oxyethylene units on average),
(LENEX698: containing 9 oxyethylene units on average). As the alkylpolyether alcohols, there may be mentioned higher alcohol polyoxyethylene ether: (Triton X-67: CA Registry No. 59030-15-8).

The microporous membrane of the fluorine-containing polymer may be rendered hydrophilic by incorporating one or two or more water-insolubilized water-soluble polymer in the porous void. Examples of the water-soluble polymer include polyvinyl alcohol, polyethylene oxide, polyethylene glycol, methyl cellulose, ethyl cellulose, hydroxyethyl cellulose, and hydroxypropyl cellulose as oxygen-containing hydrocarbons; polyacrylamide, polyvinyl pyrrolidone, polyvinylamine, and polyethyleneimine as nitrogen-containing ones; polyacrylic acid, polymethacrylic acid, and polystyrenesulfonic acid as negative charge-containing ones; and the like. The insolubilization may be effected by heat treatment, acetalization, esterification, chemical reaction with potassium bichromate, crosslinking reaction with ionized radiation, or the like. Specifically, there may be mentioned methods described in JP-B-56-2094 and JP-B-56-16187.

The microporous membrane of polysulfone can be produced by dissolving polysulfone in dioxane, tetrahydrofuran, dimethylformamide, dimethylacetamide, N-methyl-2-pyrrolidone, or a mixed solvent thereof to prepare a membrane-forming solution and pouring it onto a support or directly into a coagulating solution, followed by washing and drying. Specifically, a method disclosed in JP-A-62-27006 may be mentioned. In addition, as the microporous membrane of polysulfone, those described in JP-A-56-12640, JP-A-56-86941, JP-A-56-154051, and so forth may be also used. The microporous membrane of polysulfone can be also rendered hydrophilic by impregnation with a surfactant or incorporation of a water-insolubilized water-soluble polymer.

The other preferred non-fibrous porous membranes include brush polymer membranes made of cellulose acetates described in JP-B-53-21677, U.S. Pat. No. 1,421,341, and so forth, e.g., cellulose acetate, cellulose acetate/butyrate, or cellulose nitrate. Porous membranes made of polyamides such as 6-Nylon and 6,6-Nylon, polyethylene, polypropylene, and the like may be used. In addition, there may be also utilized porous membranes having continuous voids wherein polymer small particles, glass particles, diatomaceous earth, or the like is combined with a hydrophilic or non-water absorbable polymer, as described in JP-B-53-21677, JP-A-55-90859, and so forth.

The effective pore size of the non-fibrous microporous membrane is from 0.2 to 10 μm, preferably from 0.3 to 5 μm, particularly effectively from 0.5 to 3 μm. The effective pore size of the non-fibrous microporous membrane in the invention is shown as a pore size measured by a critical bubble pressure method (bubble point method) in accordance with ASTM F316-70. In the case that the non-fibrous microporous membrane is a membrane filter composed of a so-called brush polymer made by phase separation method, a liquid passing channel in the thickness direction is generally most narrow at the free surface side at the time when the membrane is produced, i.e., glossy surface, and thus the pore size of the cross-section of the liquid passing channel approximated to a circle is smallest near the free surface. The smallest pore size of the volume with regard to the thickness direction in the passing channel has a distribution in the plane direction of the filter and the maximum value thereof determines filtration performance toward particles. Usually, it is measured by the critical bubble pressure method.

As described above, in the membrane filter composed of a so-called brush polymer manufactured by a phase-separation method, the liquid passing channel in the thickness direction is most narrow at the free surface side at the time when the membrane is produced, i.e., glossy surface. In the case that this kind of non-fibrous microporous membrane is used as the filter material of the above blood filtration unit, the outlet side is preferably the glossy surface of the membrane filter.

To the filter material to be used in the above blood filtration unit, a third filter material can be incorporated in addition to the glass fiber filter paper and the microporous membrane. Examples of the third filter material include fibrous porous layers such as filter papers, non-woven fabrics, textile cloth (e.g., plain cloth), and knitted cloth (e.g., tricot knit). Of these, preferred are textiles, knitted fabrics, and the like. The textiles and the like may be subjected to glow discharge treatment as described in JP-A-57-66359. The third filter material is preferably arranged between the glass fiber filter paper and the microporous membrane.

Preferred microporous membrane is a polysulfone membrane, a cellulose acetate membrane, and the like. Particularly preferred is a polysulfone membrane. In the filter material used in the filtration unit, the glass fiber filter paper is arranged at the blood-supplying side and the microporous membrane is arranged at the outlet side.

The filter material to be used in the above blood filtration unit is understood to be one directed by a so-called volume filtration action, wherein blood cells are not only trapped on the surface thereof but also trapped and removed over whole length in the thickness direction of the glass fiber filter paper as they permeate in the thickness direction.

The filter material to be used in the above blood filtration unit can be integrated by adhering each layer with an adhesive placed in patches in accordance with the methods disclosed in JP-A-62-138756, JP-A-62-138757, JP-A-62-138758, JP-A-2-105043, JP-A-3-16651, and so forth.

The blood cell separation element mentioned in the above is preferably arranged between an element for injecting a specimen (in this case, whole blood) to be mentioned below and the multilayer dry assay element but the blood cell separation element may be the element for injecting the whole blood at the same time. Also in the case of a pretreatment element other than the blood cell separation element, the element can be arranged similarly and may be the element for injecting the specimen (sometimes, whole blood) at the same time, too.

{Hemolysis}

As the other specific example of the pretreatment in the invention, hemolysis may be mentioned. In the case that the specimen is whole blood and the substance to be assayed is an ingredient contained in blood cells, the destruction of blood cells, i.e., hemolysis becomes necessary. For example, in the case that the measuring target is glycohemoglobin (HbA1), it is necessary to hemolyze erythrocyte in the specimen sufficiently to solubilize hemoglobin in the erythrocyte in the solution. As the multilayer dry assay element for detecting glycohemoglobin, there is a technology using a combined product of an antibody against glycohemoglobin with an enzyme (enzyme-labeled antibody) as described in JP-A-9-166594 and JP-A-8-122335. Moreover, there is a technology of measuring glycohemoglobin by utilizing an enzyme labeled with an N-terminal glycosylated peptide of glycosylated hemoglobin β chain using the method described in JP-A-2000-310638.

In these technologies, it is also necessary to destruct blood cells and achieve hemolysis before the specimen is fed to the multilayer dry assay element. In the invention, the performance of the assay chip can be remarkably improved by achieving the hemolysis in the pretreatment element.

Moreover, the integrated assay chip of the invention may have not only the aforementioned multilayer dry assay element for detecting glycohemoglobin but also a multilayer dry assay element for detecting hemoglobin at the same time. In such a case, it becomes possible to measure the amount of hemoglobin, the amount of glycohemoglobin, and the ratio (%) of glycohemoglobin in hemoglobin by means of one integrated assay chip.

Such element for conducting hemolysis (hemolysis element) desirably has a porous character as mentioned above in the blood cell separation element. Furthermore, the hemolysis element may be formed by supporting a hemolyzing reagent in the flow channel. The hemolyzing reagent may be either a dried one or a solution.

Regarding the hemolysis treatment, for example, employable is a method of using a commercially-available hemolysis reagent or surfactant (e.g., Triton X-100), or a method of using a non-isotonic diluent for hemolysis by osmotic pressure shock. If desired, red blood cell membranes may be ultrasonically broken.

The surfactant usable for hemolysis includes anionic surfactants such as sodium dodecylsulfate (SDS) and sodium dioctylsulfosuccinate (DONS), cationic surfactants such as tetradecyltrimethylammonium bromide (TTAB) and cetyltrimethylammonium bromide (CTAB), and ampholytic surfactants such as carboxybetaine-type surfactants, as in JP-A 6-11510. Nonionic surfactants are also usable herein, including, for example, alkylphenol/polyethylene-oxide condensates such as p-(1,1,3,3-tetramethylbutyl)phenoxy-polyethoxyethanol (Triton X-100 having 9 or 10 oxyethylene units on average; Triton X-165 having 16 oxyethylene units on average; Triton X-405 having 40 oxyethylene units on average—all in Chemical Abstract Registry No. 9002-93-1); alkylphenol/polyglycidol condensates such as p-nonylphenoxy-polyglycidol (having 10 glycidol units on average); higher aliphatic alcohol/polyethylene oxide condensates such as lauryl alcohol/polyoxyethylene oxide condensates (e.g., Brij 35, in Chemical Abstract Registry No. 9002-92-0), cetyl alcohol/polyoxyethylene oxide condensates (e.g., Brij 58, in Chemical Abstract Registry No. 9004-95-9); polyethylene glycol/higher fatty acid ester condensates such as stearate/polyethylene glycol condensates (e.g., Myrj 52, Myrj 59, both in Chemical Abstract Registry No. 9004-99-3); higher fatty acid sorbitan ester/polyethylene glycol condensates such as sorbitan monolaurate/polyethylene glycol condensates (e.g., Tween 20, in Chemical Abstract Registry No. 9005-64-5).

Still another example of the pretreatment as referred to in the invention is dilution. When the method of assaying a component in a specimen is conducted by using the multilayer dry assay element based on immunoassay such as enzyme immunoassay, then the specimen must be often diluted to a predetermined ratio in advance. A dry multi-layer enzyme immunoassay method is disclosed in, for example, JP-A 5-232112. Also in the method, when the specimen is serum, it is often necessary to dilute the specimen.

In an immunoassay method for detecting minor substances in a serum specimen, diluting the specimen is effective for preventing immunoreaction with coexisting protein and for preventing the reduction in the reliability of measured data owing to non-specific adsorption. In an enzyme immunoassay method that utilizes enzymatic reaction, the concentration range (detectable range) in which quantitative determination is possible is not often broad. In such a case, a specimen is diluted whereby the concentration of the substance to be detected in the thus-diluted specimen could be within a detectable range.

Diluting a specimen may be effected generally by mixing a specimen with a diluent. For the diluent, often used is a buffer having a pH suitable for the detection reaction. For controlling the salt concentration in a specimen, salt such as sodium chloride may be added to the specimen. Any and every buffer usable for ordinary biochemical reaction may be usable herein, including, for example, phosphoric buffers, acetic buffers, carbonic buffers, boric buffers, TRIS buffers, MES buffers, HEPES buffers.

[Dilution of Specimen]

The pretreatment element may be an element for diluting specimen (dilution element). The pretreatment element may contain a diluent therein to form a dilution element. An additional element for carrying the diluent may be prepared, and it may be connected with the pretreatment element via a flow channel, whereby a specimen and the diluent may be mixed in the pretreatment element.

Preferably, the pretreatment element has a form so that a specimen and a diluent can be readily mixed therein.

[Degradation of Protein]

Still another example of the pretreatment in the invention is proteolysis. When the analyte to be assayed herein is a protein, then the protein may be previously subjected to limited degradation with a protease and then fed to the assay element. In this case, the pretreatment may be an element for degrading a protein (proteolysis element).

For example, when a saccharified protein is to be detected, then the saccharified protein in the analyte is degraded with a protease or the like, and the saccharified amino acid in the protease-processed product is further processed with a saccharified amino acid oxidase or the like, and the hydrogen oxide thus formed is detected to thereby detect the saccharified protein. The technique is described, for example, in JP-A 2001-54398 and 11-155596. Examples of the protein to be detected are saccharified albumin, saccharified globulin, saccharified hemoglobin, saccharified casein. The specimen includes, for example, blood, serum, plasma, milk, soy sauce.

The protease usable for protein decomposition in the invention may be any one capable of effectively acting on the protein contained in a specimen. For example, it includes proteases derived from animals, vegetables or microorganisms. Some examples of the proteases are mentioned below, whatsoever not limiting the invention.

Examples of animal-derived proteases are Elastase, Tripsin, Chymotripsin, Pepsin, Bovine Pancreatic Protease, Cathepsin, Calpain, Protease Type-1, Protease Type-XX (all by Sigma), Aminopeptidase M, Carboxypeptidase A (both by Boehringer Mannheim), and Pancreatin (by Wako Jun-yaku).

Examples of vegetable-derived proteases are Kallikrein, Ficin, Papain, Chimopapain, Bromelain (all by Sigma), Papain W-40, Bromelain F (both by Amano Pharmaceutical).

Examples of microorganism-derived proteases are the following (1) to (14):

(1) Bacillus-derived proteases: Subtilisin, Protease type-VIII, -IX, -X, -XV, -XXIV, -XXVII, -XXXI (all by Sigma), Thermolysin, Nagarse (both by Wako Jun-yaku), Orientase-90N, -10NL, -22BF, -Y, -5BL, Nucleisin (all by Hankyu Bioindustry), Proleather, Protease-N, -NL, -S-Amano (all by Amano Pharmaceutical), GODO-BNP, -BAP (both by Godo Shusei-sha), Protin-A, -P, Deskin, Depirays, Biosoke, Samoarse (all by Daiwa Kasei), Toyozyme NEP (by Toyobo), Neutrase, Esperase, Sabinase, Durazyme, Biofeed-Pro, Alkalase, NUE, Pillase, Clear Lens-Pro, Evalase, Nobozyme-FM, Novolan (all by Novonordisk Bioindustry), Entyron-NBS, -SA (both by Rakuto Chemical Industry), Alkali Protease GL440, Opticlean-M375 Plus, -L1000, -ALP440 (all by Ilyowa Hakko), Biopullase APL-30, SP-4FG, XL-416F, AL-15FG (all by Nagase Biochemical Industry), Aroase AP-10, Protease Y (both by Yakult Yakuhin Kogyo), Colollase-N, -7089, Belon W (all by Higuchi Shokai), Chirazyme P-1 (by Roche).

(2) Aspergillus-derived proteases: Protease type-XIII, -XIX, -XXII (all by Sigma), Sumizyme-MP-, -AP, -LP-, -FP, LPL, Enzyme P-3. (all by Shin-Nippon Chemical Industry), Orientase-20A, -ONS, -ON5, Tetrase S (all by Hankyu Bioindustry), Neurase A, Protease-A, -P, -M-Amano (all by Amano Pharmaceutical), IP enzyme, Morsin F, A0 Protease (all by Kikkoman), Protin-F, -FN, -FA (all by Daiwa Kasei), Denapsin 2P, Denazyme-SA-7, -AP, Denazyme AP (all by Nagase Biochemical Industry), Protease YP-SS, Pantidase-NP-2, -P (all by Yakult), Sakanase (by Kaken Pharma), Flavorzyme (by Novonordisk Bioindustry), Belon PS (by Higuchi Shokai).

(3) Rhisopus-derived proteases: Protease Type-XVIII (by Sigma), Peptidase R, Neurase F (both by Amano Pharmaceutical), XP-415 (by Nagase Biochemical Industry).

(4) Penicillium-derived proteases: PD enzyme (by Kikkoman).

(5) Streptomyces-derived proteases: Protease Type-XIV (Pronase-XXI) (by Sigma), Actinase-AS, -AF (both by Kaken Pharma), Tasinase (by Kyowa Hakko), Alkalofilic Proteinase (by Toyobo).

(6) Staphylococcus-derived proteases: Protease-Type XVII (by Sigma).

(7) Clostridium-derived proteases: Clostripain, Nonspecific Neutral Protease (both by Sigma).

(8) Lysobacter-derived proteases: Endoproteinase Lys-c (by Sigma).

(9) Grifola-derived proteases: Metalloendopeptidase (by Sigma).

(10) Yeast-derived proteases: Proteinase A (by Sigma), Carboxypeptidase Y (by Boehringer Mannheim).

(11) Tritirachium-derived proteases: Proteinase K (by Sigma).

(12) Thermus-derived proteases: Aminopeptidase T (by Boehringer Mannheim).

(13) Pseudomonus-derived proteases: Endoproteinase Asp-N (by Wako Jun-yaku).

(14) Achromobacter-derived proteases: Lysyl Endopeptidase, Achromopeptidase (both by Wako Jun-yaku).

In the proteolysis step, protease as above is often used. The type of protease to be used is not specifically defined. For example, protease K, subtilisin, trypsin, aminopeptidase, saccharified peptide-protease and the like may be used.

The enzyme for use in proteolysis may be directly fixed to the proteolysis element or may be contained in a dry state. In addition, as another preferred example, the enzyme may be contained in the proteolysis element after supported on the fine particles as mentioned above.

The method for fixing the enzyme to the pretreatment element in the assay chip is not particularly limited and the fixing can be achieved by any of a conjugate bond method, a physical adsorption method, an ionic bond method, and the like. In particular, in the case that the assay chip of the invention is in a microchip form, it is also preferable to utilize the technology described in JP-A-2004-125406, for example. In addition, it is also preferable to support the enzyme on beads or fine particles as described in JP-A-2004-61496.

{Denaturation of Protein}

As the other specific example of the pretreatment in the invention, there may be mentioned the denaturation of a protein. In the case that an assay of an ingredient in the specimen is carried out using a multilayer dry assay element based on the detection method utilizing an antigen-antibody binding reaction such as immunoassay, it is essential to combine a target protein in the specimen with an antibody reagent. However, since a segment (epitope) which combines with an antibody in the protein is present inside the protein, there frequently arises a problem that the reaction with the antibody reagent does not rapidly proceed. The assay chip of the invention may have an element having a protein denaturant (protein denaturation element) as a pretreatment element. The protein denaturant contained in the protein denaturation element is added to the specimen to denature a protein, whereby the epitope is exposed onto the protein surface and hence the antigen-antibody reaction can be accelerated. Examples of the protein denaturant include chaotropic reagents, surfactants, organic solvents, and the like.

{Removal of Endogenous Substance}

As the other specific example of the pretreatment in the invention, there may be mentioned the removal of an endogenous substance. In the case that the specimen is, for example, serum or whole blood, an ingredient contained in the specimen (endogenous substances) frequently influences the detection of the objective ingredient and thus, there arises necessity to remove the endogenous substance or to lower the activity before the specimen is fed to the assay element.

The pretreatment element may be an element for removing the endogenous substance.

In the case that the endogenous substance is an enzyme, the element for removing the endogenous substance may retain an inhibitor which selectively acts on the enzyme. By inhibiting the activity of the enzyme with the inhibitor, there is achieved the same effect as in the case that the enzyme is removed.

For example, in enzyme immunoassay, in the case that the enzyme to be utilized for detection (labeling enzyme) is also present in the specimen, there is a case that the endogenous enzyme may severely inhibit the detection of the reaction. In the case of enzyme immunoassay wherein amylase of Bacillus subtilis is used as the labeling enzyme as described in JP-A-1-237455, JP-A-1-321360, and JP-A-1-321361, the amylase present in serum frequently lowers accuracy of the. detection. In order to prevent such a phenomenon, by reacting the specimen with an inhibitor specific to the amylase in serum, the influence of the endogenous amylase can be eliminated.

Moreover, in the case that the endogenous substance is an enzyme substrate, the element for removing the endogenous substance can retain the enzyme and decompose the enzyme substrate by the enzyme. Furthermore, the element for removing the endogenous substance can retain a specifically adsorptive substance such as an antibody to remove the enzyme substrate.

For example, ascorbic acid (and derivatives thereof) present in serum sometimes remarkably influences the oxidation-reduction coloring system of the assay element. In such a case, it is necessary to remove endogenous ascorbic acid beforehand. Mainly ascorbic acid oxidase is employed for the removal of ascorbic acid and the methods described in JP-A-9-089867, JP-A-07-303497, and JP-A-1 1-309466 can be utilized.

The element for removing the endogenous substance necessarily contains a reagent for removing the endogenous substance itself or its activity. Such a reagent may be directly fixed to the pretreatment element or may be contained in a dry state. Moreover, as the other preferred example, it may be contained in the pretreatment element after supported on fine particles.

The method for fixing the enzyme to the pretreatment element in the assay chip is not particularly limited and a known technology may be employed. For example, the fixing can be achieved in the same manner as mentioned above in the paragraph of {Decomposition of protein}. It is also preferred to support it on beads or fine particles.

{Antigen-Antibody Reaction}

Still other specific example of the pretreatment in the invention includes an antigen-antibody reaction. At assaying a component in a specimen, the assay means may vary depending on the cases that the component is an antigen or an antibody in an antigen-antibody reaction, but the component can be assayed in both cases.

In the specimen, in the case that the component to be assayed is an antigen, the amount of the trapped antigen can be assayed by fixing, to the pretreatment element, a substance such as fine particles, mesh, pillar, and a porous substance on which an antibody is supported or directly supporting the antibody on a member of the pretreatment element to trap the antigen within the pretreatment element and subsequently by assaying the amount of the antigen which cannot be trapped or removing the trapped antigen from the inside of the pretreatment element in a following step. Moreover, use may be also made of a method of determining the amount by supporting an antibody-enzyme composite on the pretreatment element and converting the amount of the antigen in the specimen into an enzymatic activity. Furthermore, use may be also made of a method of determining the amount by supporting an antigen or antigen analog (antigen analogous substance)-enzyme composite on the pretreatment element and introducing a mixture of an antigen-containing liquid and the specimen into the pretreatment element to convert the amount of the antigen in the specimen into an enzymatic activity.

To the contrary, the component to be assayed in the specimen is an antibody, the amount of the antibody in the specimen can be determined using the pretreatment element wherein the antigen and the antibody is reversed in the above pretreatment element.

[Multilayer Dry Assay Element]

In the invention, a multilayer dry assay element is used as the detection system of the integrated assay chip. The multilayer dry assay element contains a reagent for detecting an ingredient in a specimen. The multilayer dry assay element is an assay element which employs so-called dry chemistry. In the invention, by using the multilayer dry assay element as the detection system of the integrated assay chip, rapid detection becomes possible since the reagent is in a dry state and stable and the reaction proceeds with only the water content of the specimen.

The multilayer dry assay element in the invention means a dry assay element wherein all or part of reagents necessary for qualitative or quantitative assay of an ingredient to be measured in the specimen are incorporated in one or more layers. It is an assay element using a so-called dry chemistry. Specifically, examples of such multilayer dry assay element include those described in Fuji Film Research Report, No. 40, p. 83 (Fuji Photo Film Co., Ltd., published in 1995), Rinsho Byori, special number, special topic No. 106, Dry Chemistry/Kan-i Kensa no Aratanaru Tenkai (Rinsho Byori Kankou Kai, published in 1997), and so forth.

The aforementioned multilayer dry assay element usually contains at least one functional layer. The number of the functional layer is not particularly limited as far as the number is one or more, and the element may be one-layered or may have plurality of layers, i.e., two or more layers.

Specific examples of the functional layer include a spreading layer, an adhesive layer which adheres the spreading layer and a functional layer other than the spreading layer, a water-absorbing layer which absorbs a liquid reagent, a mordanting layer which prevents the diffusion of a dye formed by a chemical reaction, a gas permeation layer which allows to permeate gas selectively, an intermediary layer which suppresses or accelerates substance transfer between the layers, a light-shielding layer for stably conducting reflective photometry, a color-shielding layer which suppresses the influence of an endogenous dye, a reagent layer containing a reagent which reacts with a target substance to be assayed, a coloring layer containing a coloring agent, and the like. They can be suitably selected as occasion demands.

As one example of the multilayer dry assay element, a hydrophilic polymer layer can be, for example, provided on a support as a functional layer, optionally via the other layer such as an undercoat layer. The hydrophilic polymer layer is, for example, a non-porous water-absorbing and water-permeable layer, and a water-absorbing layer basically composed of the hydrophilic polymer layer alone, a reagent layer containing all or part of coloring agents directly participating in the coloring reaction using the hydrophilic polymer as a binder, a detection layer containing an ingredient (e.g., mordant).which fixes and immobilizes the colored dye in the hydrophilic polymer, and the like can be provided.

(Spreading Layer)

The spreading layer of the multilayer dry assay element is a layer which takes an action (called as spreading action, extending action, or metering action) to feed, to the water-absorbing reagent layer or the water-absorbing layer, an aqueous liquid sample [e.g., blood (whole blood, plasma, serum), lymph fluid, saliva, cerebrospinal fluid, vaginal fluid, urine, drinking water, juice, liquor, river water, factory waste water, etc.] which is fed by dropping to the upper surface (a surface remote from the support), with extending in the lateral direction without substantially unevenly distributing the ingredients contained in the aqueous liquid sample in a ratio of nearly constant volume per unit area.

A porous spreading layer is preferred, and there may be mentioned, for example, a non-fibrous isotropic fine porous medium layer which is represented by the membrane filter described in JP-A-49-53888 and so forth, a non-fibrous porous spreading layer which is represented by the continuous void-containing three dimensional lattice particulate structure layer wherein polymer microbeads are adhered to each other in a point contact form with a water-non-swelling adhesive described in JP-A-55-90859 and so forth, a porous spreading layer composed of a textile fabric described in JP-A-5-164356, JP-A-57-66359, and so forth, a porous spreading layer composed of a knitted fabric described in JP-A-60-222769, and so forth, and the like.

(Reagent Layer)

The reagent layer is a water-absorbing and water-permeable layer in which at least a part of a reagent composition capable of reacting with a an analyte in an aqueous liquid specimen to thereby undergo an optically detectable change is substantially uniformly dispersed in a hydrophilic polymer binder therein. The reagent layer includes an indicator layer and a colorant layer.

The hydrophilic polymer usable as the binder in the reagent layer is generally a natural or synthetic hydrophilic polymer of which the degree of swelling with water falls between about 150% and about 2000% at 30° C., preferably between about 250% and about 1500%. Examples of the hydrophilic polymer of the type are gelatin (e.g., acid-processed gelatin, deionized gelatin), gelatin derivatives (e.g., phthalated gelatin, hydroxyacrylate-grafted gelatin), agarose, pullulan, pullulan derivatives, polyacrylamide, polyvinyl alcohol, polyvinylpyrrolidone, as in JP-A 60-108753.

The reagent layer may be suitably crosslinked and cured with a crosslinking agent. Examples of the crosslinking agent are, for example, known vinylsulfone-type crosslinking agents such as 1,2-bis(vinylsulfonylacetamido)ethane and bis(vinylsulfonylmethyl) ether, and aldehydes for gelatin; and aldehydes and epoxy compounds containing two glycidyl groups for methallyl alcohol copolymer.

Preferably, the dry thickness of the reagent layer falls between about 1 μm and about 100 μm, more preferably between about 3 μm and about 30 μm. Also preferably, the reagent layer is substantially transparent.

For the reagent to be in the reagent layer and in any other layers of the multilayer dry assay element, any one suitable for the intended detection may be selected in accordance with the substance to be detected therewith.

(Light-Shielding Layer)

If desired, a light-shielding layer may be provided on the reagent layer. The light-shielding layer is a water-pervious or water-permeable layer in which light-absorbing or light-reflecting (the two are referred to as light-shielding) fine particles are dispersed in and held by a small amount of a film-forming hydrophilic polymer binder therein. The function of the light-shielding layer is as follows: When a detectable change (color change, color formation) having occurred in the reagent layer is observed on the light-pervious support side in a mode of reflective light observation, then the light-shielding layer may shield the color of the aqueous liquid specimen itself spotwise applied to the spreading layer to be mentioned hereinunder, especially the red color of hemoglobin in a whole blood specimen applied thereto. In addition, the light-shielding layer serves also as a light-reflection layer or a background layer.

Examples of the light-reflecting fine particles are titanium dioxide fine particles (rutile-type, anatase-type or brookite-type microcrystal particles having a particle size of from about 0.1 μm to about 1.2 μm), barium sulfate fine particles, aluminium fine particles and fine flakes. Examples of the light-absorbing fine particles are carbon black, gas black, carbon microbeads. Of those, preferred are titanium dioxide fine particles and barium sulfate fine particles. More preferred are anatase-type titanium dioxide fine particles.

Examples of the film-forming hydrophilic polymer are the same hydrophilic polymers as those usable in producing the above-mentioned reagent layer, as well as weakly-hydrophilic regenerated cellulose, and cellulose acetate. Of those, preferred are gelatin, gelatin derivatives, and polyacrylamide. Gelatin and gelatin derivatives may be used, as mixed with any known curing agent (crosslinking agent).

The light-shielding layer may be provided on the reagent layer by applying an aqueous dispersion of light-shielding fine particles and a hydrophilic polymer onto the reagent layer and drying it thereon in any known method. In place of providing the light-shielding layer, light-shielding fine particles may be incorporated into the above-mentioned spreading layer.

(Adhesive Layer)

An adhesive layer may be provided on the reagent layer optionally via a layer such as a light-shielding layer, in order that a spreading layer could be stuck to and laminated on the reagent layer.

Preferably, the adhesive layer is formed of a hydrophilic polymer, which, while wetted with water or swollen with water, can bond a spreading layer whereby the constitutive layers are therefore integrated. Examples of the hydrophilic polymer usable for forming the adhesive layer may be the same hydrophilic polymers as those used in forming the reagent layer. Of those, preferred are gelatin, gelatin derivatives and polyacrylamide. The dry thickness of the adhesive layer is generally from about 0.5 μm to about 20 μm, preferably from about 1 μm to about 10 μm.

Such an adhesive layer may be provided not only on the reagent layer but also on any other desired layer for enhancing the interlayer adhesion power between the constitutive layers. The adhesive layer may be provided in any known method of applying an aqueous solution that contains a hydrophilic polymer and optionally surfactant and others onto a support or a reagent layer.

(Water-Absorbing Layer)

A water-absorbing layer may be provided between the support and the reagent layer in the multilayer dry assay element. The water-absorbing layer is a layer comprising, as the main ingredient thereof, a hydrophilic polymer that absorbs water and swells, and this absorb water of an aqueous liquid specimen having reached the interface of the water-absorbing layer or having penetrated through the layer. When the assay chip is used particularly for a whole blood specimen, then the water-absorbing layer acts to promote penetration of the aqueous liquid component, plasma to the reagent layer. The hydrophilic polymer for the water-absorbing layer may be selected from those mentioned hereinabove for the reagent layer. In general, gelatin or gelatin derivatives, polyacrylamide, polyvinyl alcohol, especially the above-mentioned gelatin or deionized gelatin are preferred for the water-absorbing layer. Like the reagent layer, the above-mentioned gelatin is most preferred for the water-absorbing layer. The dry thickness of the water-absorbing layer may be from about 3 μm to about 100 μm, but preferably from about 5 μm to about 30 μm; and the coating amount thereof may be from about 3 g/m² to about 100 g/m², but preferably from about 5 g/m² to about 30 g/m². The water-absorbing layer may contain a pH buffer such as that mentioned below, as well as a known basic polymer or the like, whereby the pH of the layer may be controlled during in service (while used for assay operation). Further, the water-absorbing layer may contain a known mordant agent, a polymer mordant agent, etc.

(Detection Layer)

The detection layer is a layer in which the dye formed in the presence of the component to be detected diffuses and it is optically detected via a light-transmissive support. This is formed of a hydrophilic polymer. This may contain a mordant agent, for example, a cationic polymer for anionic dye. The water-absorbing layer as mentioned above is, in general, a layer in which the dye formed in the presence of the component to be detected does not substantially diffuse. In this point, therefore, the water-absorbing layer is differentiated from the detection layer.

The multilayer dry assay element may be prepared and produced by any known method. In its use, the element may be cut into small squares having a size of from about 1 mm² to about 30 mm², or into small discs having the same size as that of the squares. If desired, it may be cut into smaller ones.

A large number of such multilayer dry assay elements have been developed and commercialized, and FUJI DRI-CHEM (by Fiji Photo Film) is one example. In the invention, such a multilayer dry assay element may be used directly as it is; or a part of it may be used.

So far as the multilayer dry assay element is kept in contact with at least one (3) flow channel, the multilayer dry assay element may be in any form where the element is connected with the flow channel or is built in the flow channel. When a plurality of multilayer dry assay elements are used in one assay chip, they may be in one site where they are connected with each other via a flow channel, or may be separately arranged.

In many cases, a multilayer dry assay element often has a spreading layer as the uppermost layer thereof where blood or its component is developed in the horizontal direction. In the invention, however, such a spreading layer is not always necessary.

[Flow channel]

In the first embodiment of the assay chip of the invention, a flow channel for connecting the multilayer dry assay element and the pretreatment element is contained. Accordingly, the width of the flow channel may be broad or narrow, depending on the necessity thereof However, when the amount of the specimen. is small, then the flow channel is preferably a micro-flow channel (having an equivalent diameter of 3 mm or less).

The equivalent diameter as referred to herein is a technical term generally used in the field of mechanical engineering. Briefly, a circular pipe that is equivalent to a pipe (flow channel in the invention) having an unspecified cross-sectional profile is given to the unspecified pipe, and the diameter of the equivalent circular pipe is referred to as an equivalent diameter of the unspecified pipe. The equivalent diameter, deq is defined as follows: deq=4A/p, in which deq means the equivalent diameter of the pipe, A means the cross section of the pipe, and p means the wetted perimeter (peripheral length) of the pipe. When applied to a circular pipe, the equivalent diameter is equal to the diameter of the circular-pipe. The equivalent diameter is used for presuming the fluidity or thermal conduction characteristics in the pipe, based on the data of the equivalent circular tube, and it indicates the spatial scale (typical length) of a phenomenon. The equivalent diameter of a square having a side “a” is deq=4a²/4a=a. The flow between parallel flat plates having a channel height h is deq=2h. Its details are described in Dictionary of Mechanical Engineering (edited by the Mechanical Society of Japan, 1997, Maruzen).

The equivalent diameter of the micro-flow channel used in the invention is 3 mm or less, but preferably from 10 to 1000 μm, more preferably from 20 to 500 μm.

Not specifically defined, the length of the flow channel is preferably from 1 mm to 10000 mm, more preferably from 5 mm to 100 mm.

The width of the flow channel for use in the invention is preferably from 1 to 3000 μm, more preferably from 10 to 2000 μm, even more preferably from 50 to 1000 μm. When the width of the flow channel falls within the defined range, then it is favorable since the flowability of the specimen such as blood lowers little owing to the wall pressure given to the specimen, and, in addition, the necessary amount of the specimen may be reduced.

One alone or two or more branched flow channels may be in the integrated assay chip, depending on the number of the elements to be disposed in the assay chip. The flow channel may have any form such as be straight or curved, but it is preferably straight.

A specimen moves from the flow channel to the multilayer dry assay element. For handling the specimen, or that is, the fluid in the flow channel, preferably employed is a continuous flow system, a liquid droplet (liquid plug) system, a driving system, as well as a method of utilizing a capillary phenomenon or utilizing pressure.

[Other Elements]

The assay chip in the invention may have the element for injecting a specimen such as whole blood, as mentioned above. Moreover, the element may be connected to or incorporated into the pretreatment element or the flow channel.

The element for injecting whole blood means a guide capable of injecting whole blood into the above assay chip, and may be any form as far as it can inject whole blood. The element for injecting whole blood may have a detachable blood-sampling element and the element may be in a needle form. The above guide and the blood-sampling needle may be combined.

As the method for combining the guide and the blood-sampling needle, the same methods as those mentioned as the combining technologies usable at the assembly of the assay chip can be employed.

The blood-sampling needle in the invention samples blood from blood vessel and introduces it into the assay chip of the invention. For example, it may be a needle like a usual needle for injection syringe or may be smaller one in view of a minute amount of blood sampling. Moreover, it is preferable to alleviate pain at the blood sampling by thinning the needle point. Furthermore, it is also possible to manufacture the needle utilizing the aforementioned micro-fabrication technology.

The material composing the blood-sampling needle is usually a metal and examples thereof includes materials used for so-called needles for injection, such as stainless steels, nickel-titanium alloys, and tungsten. In addition, it is also possible to use resins such as plastics mentioned above as materials composing the cartridge. Specifically, there may be mentioned PCO, PS, PC, PMMA, PE, PET, PP, PDMS, and the like.

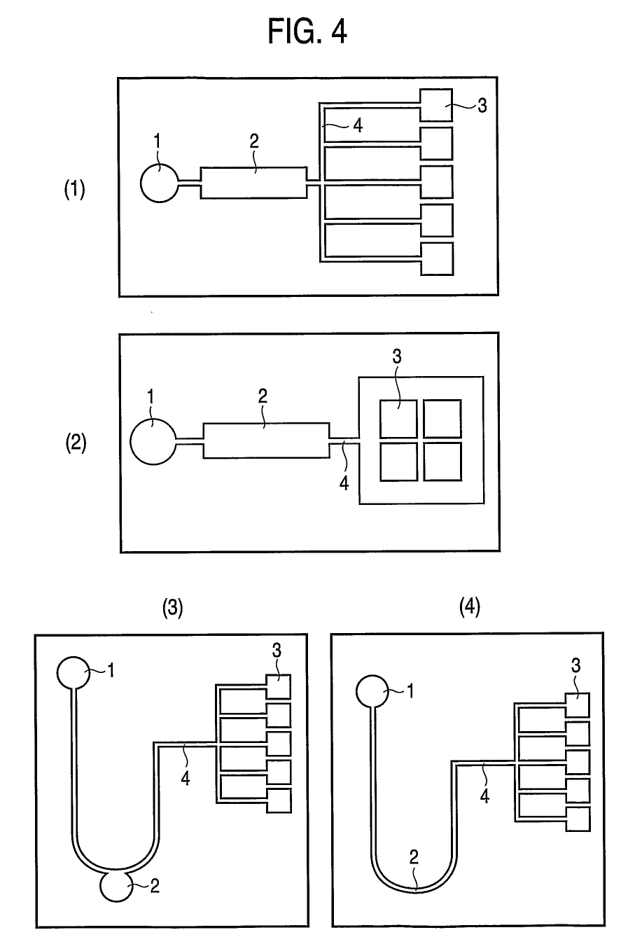

The arrangement of the pretreatment element, the multilayer dry assay element, and the flow channel of the assay chip of the invention can be, for example, explained by the schematic diagram using a blood cell-separating element as a pretreatment element as shown in FIG. 4, but is not limited thereto.

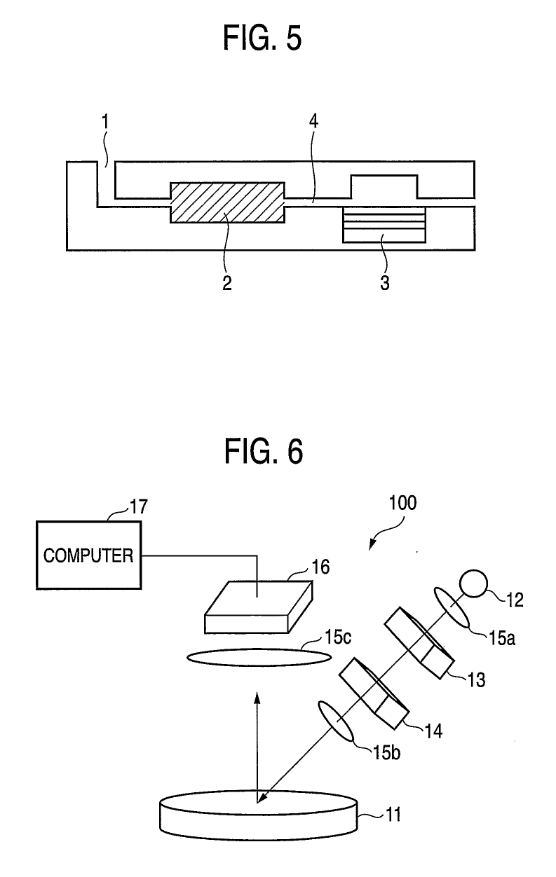

Moreover, the cross-section of the assay chip of the invention is, for example, illustrated in a typical view shown in FIG. 5, but is not limited thereto.

[Specimen]

The substances to be assayed which are targets of the assay chip of the invention are not particularly limited and a specific ingredient in any liquid sample (e.g., a body fluid such as whole blood, plasma, serum, lymph fluid, urine, saliva, cerebrospinal fluid, or vaginal fluid; drinking water, juice, liquor, river water, factory waste water, or the like). For example, albumin (ALB), glucose, urea, bilirubin, cholesterols, proteins, enzymes [e.g., enzymes in blood such as lactate dehydrogenase, CPK (creatine kinase), ALT (alanine aminotransferase), AST (aspartate aminotransferase), and GGT (γ-glutamyl transpeptidase)] can be assayed.

To the assay chip of the invention, an aqueous liquid sample solution, which is a specimen in the range of, for example, 0.1 to 30 μl, preferably 1 to 10 μl, is injected or point-attached. The specimen is passed through the pretreatment element and the multilayer dry assay element in this order or, in the case that a flow channel is provided, through the pretreatment element, the flow channel, and the multilayer dry assay element in this order. The excess liquid residue contained in the specimen is absorbed in the water-absorbing layer provided in the multilayer dry assay element as occasion demands. For the transfer of the specimen, the method described in the above paragraph of [Flow channel] is utilized. The passing through respective elements may be achieved by respective different methods or one method. After the passing, the assay chip is incubated at a constant temperature in the range of about 20° C. to about 45° C., preferably about 30° C. to about 40° C. for 1 to 10 minutes. The coloring or change in the multilayer dry assay element is measured by reflection photometry from the light-transmittable support side and the amount of the substance to be assayed in the specimen can be determined based on the principle of colorimetry using a calibration curve prepared beforehand. Moreover, it is also possible to apply photometry as occasion demands.

[Photometry]

The measurement by the assay chip in the invention can be performed by using an area sensor.

Hereinafter, an outline of the configuration of a measuring apparatus using an area sensor is described by referring to FIG. 6.

A measuring apparatus 100 comprises an assay chip setting portion 11, in which a specimen to be measured is set, and a light source 12 employing a light emitting device, such as a halogen lamp, for irradiating light onto the specimen, a light variant portion 13 for changing the intensity of light irradiated from the light source 12, a wavelength tunable portion 14 for changing the wavelength of light irradiated from the light source 12, lenses 15a and 15b for converting light rays irradiated from the light source 12 into parallel light rays and for condensing the light irradiated therefrom, a lens 15c for condensing reflection light reflected from the specimen, an area sensor 16 serving as a light receiving device for receiving the reflection light condensed by the lens 15c, and a computer 17 for controlling each of such portions, for obtaining results of measurements according to the state of the light variant portion 13 and to an amount of light received by the area sensor 16, and for outputting the obtained results to a display or the like. Incidentally, although the computer 17 is adapted to control each of the portions in this embodiment, a computer serving as an integrated controller for controlling each of the portions may be provided separately from the computer 17.

An assay chip is provided in the assay chip setting portion 11. A portion actually devoted to the measurement is the multilayer dry assay element in the assay chip, which reacts with the specimen.

The light variant portion 13 is adapted to change the intensity of light, which is irradiated onto the specimen from the light source 12, by mechanically putting a perforated or meshed plate member made of metal, such as stainless steel, and an attenuating filter, such as a neutral density filter, in and out of the space provided between the light source 12 and the specimen. In the initial setting thereof, this attenuating filter is inserted therebetween. Incidentally, in the following description, it is assumed that the meshed metal plate is a meshed stainless steel plate. Further, the perforated or meshed stainless steel plate member and the attenuating filter, such as the ND filter, may manually be put in and out of the space.

The wavelength tunable portion 14 is adapted to change the wavelength of light, which is irradiated onto the specimen from the light source 12, by mechanically putting one of plural kinds of interference filters in and out of the space provided between the light source 12 and the specimen. Incidentally, although the wavelength tunable portion 14 is set between the light variant portion 13 and the assay chip setting portion 11 in this embodiment, the wavelength tunable portion 14 may be set between the light source 12 and the light variant portion 13. Additionally, the wavelength variant portion 14 may be adapted so that plural kinds of interference filters can manually be put in and out of the space provided therebetween.

The area sensor 16 is a solid-state imaging device, such as a CCD, and operative to receive reflection light obtained from light irradiated from the light source 12 when the reagent in the multilayer dry assay element, which is set in the assay chip setting portion 11, reacts with the specimen, such as blood, and also operative to convert the received light to an electrical signal and to output the electrical signal to the computer 17. The area sensor 16 can receive the light reflected by the multilayer dry assay element correspondingly to each of areas thereof Thus, the measurement of light from areas thereof, which are respectively associated with the reagents, can simultaneously be performed, that is, the measurements respectively associated with plural items can be performed.

The computer 17 is operative to convert an electrical signal, which is outputted from the area sensor 16 and has a level corresponding to the amount of received light, into an optical density value according to data of a calibration curve, which is preliminarily stored in an internal memory, and also operative to obtain the contents of various components, which are contained in the specimen, according to the optical density value and also operative to output the obtained contents of the components to the display or the like. In the case of measuring plural items, the computer 17 extracts electrical signals, whose levels correspond to the amount of received light outputted from the area sensor 16, corresponding to plural areas of the multilayer dry assay element, respectively, and obtains the contents of the components contained in the specimen, which are respectively associated with the plural areas. Further, the computer 17 controls the light variant portion 13 and the wavelength tunable portion 14 according to the amount of light reflected by the specimen, which is received by the area sensor 16, and to the kinds of the reagents to be reacted with the specimen, in such a way as to change the amount of light irradiated from the light source 12 and the wavelength of this light.

In a case where the amount of light reflected from the specimen is so small to such an extent that this amount is not within the dynamic range of the area sensor 16, in the measuring apparatus 100 of the aforementioned configuration, the meshed stainless steel plate or the ND filter is detached from the space between the light source 12 and the specimen. The light variant portion 13 increases the intensity of light irradiated from the light source 12. Consequently, the amount of light reflected from the specimen is increased in such a way as to be within the dynamic range of the area sensor 16. Thus, even in a case where the dynamic range of the area sensor 16 is narrow, the reflection light can be received with good precision. The accuracy of measurement of the contents of components included in the specimen is enhanced.

Further, in a case where the multilayer dry assay element containing, for example, four kinds of reagents A, B, C, and D, the measuring apparatus 100 obtains the amount of light reflected from each of the rears containing the reagents A to D. In a case where one of the amounts of light is not within the dynamic range of the area sensor 16, the light variant portion 13 causes the meshed stainless steel plate member or the ND filter to be inserted and taken out every constant time. Furthermore, because the wavelengths of light rays reflected from the areas differ from one another, the wavelength tunable portion 14 changes over the plural interference filters according to the wavelengths.

The flowing description describes, for example, a case where the amounts of light reflected from the areas containing the reagents A and B are so small to the extent that these amounts are not within the dynamic range of the area sensor 16, where the amounts of light reflected from the areas containing the reagents C and D are within the dynamic range of the area sensor 16, and where the wavelengths of light rays, which are outputted when the reagents A to D react with blood, differ from one another.

In this case, in the measuring apparatus 100, the light source 12 irradiates light onto the multilayer dry assay element. Light rays reflected from the areas of slides are received by the area sensor 16. The computer 17 decides whether the amount of light reflected from each of the areas is within the dynamic range of the area sensor 16. In this case, the amount of light reflected from each of the areas respectively containing the reagents A and B is small to the extent that this amount of reflected light is not within the dynamic range of the area sensor 16. After light is irradiated for a certain time from the light source 12, the computer 17 controls the light variant portion 13 so that the ND filter is detached from between the light source 12 and the specimen. The light is irradiated for the certain time in this state. Thereafter, the computer 17 controls the light variant portion 13 so that the ND filter is inserted between the light source 12 and the specimen. Such an operation is repeated. Thus, plural kinds of components to be measured can be measured with good accuracy by the single multilayer dry assay element.

The computer 17, which thus controls the light variant portion 13, also controls the wavelength tunable portion 14 according to the kinds of the reagents A to D simultaneously, so that the wavelength tunable portion 14 changes over four kinds of interference filters in turn. During the light variant portion 13 causes the ND filter to be detached, the wavelength tunable portion 14 switches the interference filter associated with the reagent A and the interference filter associated with the reagent B to each other. During the light variant portion 13 causes the ND filter to be inserted, the wavelength tunable portion 14 switches the interference filter associated with the reagent C and the interference filter associated with the reagent D to each other. Consequently, even in a case where the wavelength of light rays outputted from the plural kinds of components contained in the specimen differ from one another, the contents of the plural kinds of components to be measured, which are contained in the specimen, can be measured by the single multilayer dry assay element.

Even in the case of using the CCD, whose dynamic range is narrow, the measuring apparatus 100 can achieve high-precision measurement by changing the intensity of light irradiated from the light source 12. However, similarly, the high-precision measurement can be performed by changing the exposure time (the time, during which the reflection light is received) of the CCD under the control of the computer 17 without changing the intensity of light.

Incidentally, although light is irradiated from the light source 12 to the specimen and the contents of components contained in the specimen are found from the light reflected therefrom in this embodiment, the contents of components contained in the specimen may be found from light transmitted by the specimen.

Further, although the light reflected from the specimen is received by using the area sensor, such as the CCD, in this embodiment, such a light receiving device according to the invention is not limited to the area sensor. A line sensor may be used instead of the area sensor.

Additionally, preferably, the CCD used in this embodiment is a CCD of the honeycomb type, in which light receiving portions, such as photodiodes, are arranged at predetermined intervals lengthwise and breadthwise on a semiconductor substrate, and in which the light receiving portions included in one of each pair of the adjacent light-receiving-portion columns are disposed in such a way as to be shifted from the light receiving portions included in the other adjacent light-receiving-portion column by about half the pitch of the light receiving portions in each of the light-receiving-portion columns in the direction of the light-receiving-portion column.

Although it has been described in the foregoing description that the measuring apparatus 100 changes the intensity of light in real time according to the amount of light reflected from the specimen, each of the contents of the components to be measured may be measured in a preset sequence corresponding to the component to be measured, which is contained in the specimen. Operations in this case are described hereinbelow.

When the assay chip is set in the assay chip setting portion 11, and the item to be measured is set therein, the measuring apparatus 100 starts measuring this item by using a pattern associated with this item to be measured. First, the computer 17 selects the intensity of light, which is utilized for the measurement, from plural kinds of intensities. Then, light having the selected intensity is irradiated to the specimen. When the area sensor 6 receives reflection light reflected from the specimen, the computer 17 outputs a measurement result according to both the amount of the reflection light received by the area sensor 16 and the selected intensity of light. This sequence of operations enables a good-precision measurement of the component to be measured, which is contained in the specimen.

In the case of changing the exposure time of the CCD without changing the intensity of light, when the assay chip is set in the assay chip setting portion 11, and the item to be measured is set therein, the measuring apparatus 100 starts measuring this item by using a pattern associated with this item to be measured. First, the computer 17 causes light to be irradiated to the specimen. Then, the area sensor 16 receives reflection light reflected from the specimen for the exposure time selected by the computer 17. Finally, the computer 17 outputs a measurement result according to both the amount of the reflection light received by the area sensor 16 and the selected intensity of light. This sequence of operations enables good-precision measurement of the component to be measured, which is contained in the specimen.

As described above, the measuring apparatus 100 causes the light source 12 to irradiate light to the multilayer dry assay element in the assay chip, and obtains the contents of the component contained in the specimen from resultant reflection light or transmitted light. However, the operation of obtaining the contents by the measuring apparatus 100 is not limited thereto. The measuring apparatus 100 may obtain the contents of the component contained in the specimen by detecting light, such as fluorescence, emitted from the multilayer dry assay element when light is irradiated to the multilayer dry assay element from the light source 12. Alternatively, the measuring apparatus 100 may the contents of the component contained in the specimen by causing the light variant portion 13 to completely shut out light irradiated from the light source 12 or by inhibiting the use of the light source 12 to thereby establish a state, in which light is not irradiated to the multilayer dry assay element at all, and by then detecting light, such as chemiluminescence, emitted from the reagent supporting portion.

Regarding the measurement operation with it, the assay chip of the invention enables high-accuracy quantitative assay in an extremely simplified manner by the use of chemical assay devices such as those in JP-A 60-125543, 60-220862, 61-294367, 58-161867 (corresponding to U.S. Pat. No. 4,424,191). Depending on the object and the necessary accuracy, the degree of coloration may be judged with the naked eye for semi-quantitative determination.

The assay chip of the invention is stored and reserved in dry before used for assay, and therefore does not require preparing a reagent for its use. In addition, the stability of a reagent is generally kept higher in dry, and therefore the assay chip of the invention is superior to a wet process where a reagent solution must be prepared every time when it is used, in point of the simplicity and the rapidity in using it. Moreover, another superiority of the assay chip of the invention is that it is a detection system that enables rapid and high-accuracy examination by the use of a minor amount of a liquid analyte specimen. The assay chip of the invention does not require a complicated pretreatment.

The invention is described more concretely with reference to the following Examples, which, however, are not intended to restrict the scope of the invention.

### EXAMPLES

### Production Example 1

Formation of PDMS Depression Pattern:

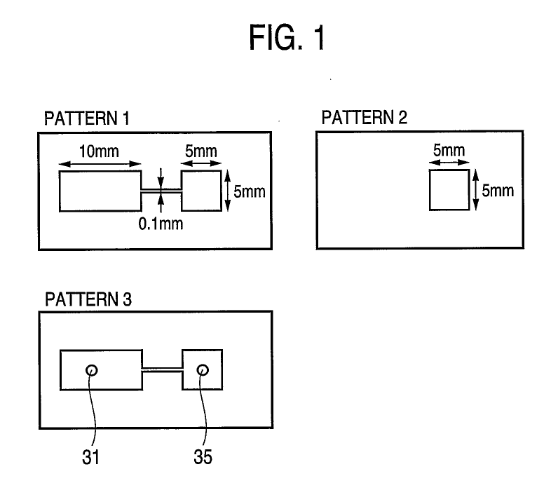

A thick photoresist film of SU-8 having a thickness of 100 μm was formed on a silicon wafer in a mode of spin coating. This was preheated at 90° C. for 1 hour, and then exposed to UV light through a mask having a flow channel pattern (1) as in FIG. 1, and the exposed area was cured at 90° C. for 1 hour. The uncured area was dissolved and removed with propylene glycol monomethyl ether acetate (PGMEA), then washed with water and dried. This is used as a silicon wafer/SU8 projection pattern.

PDMS (DuPont Sylgard/curing liquid=10/1 mixture liquid) was cast onto the silicon wafer projection pattern, and cured at 80° C. for 2 hours. Then, this was gently peeled away from the silicon wafer projection pattern, and a PDMS depression pattern (1) as in FIG. 1 was thus formed. Using a bio-laboratory trephine (by Kai Industry), an inlet for introducing specimen 31 and an air extractor 35 (diameter, 1 mm) were formed (pattern (3)).

Next, a PET base having a thickness of 0.5 mm was stuck to the silicon wafer with an instant adhesive to thereby form a projection pattern for the flow channel pattern (2) as in FIG. 1. According to the method mentioned above, PDMS was cast onto it, and a PDMS depression pattern (2) was thus formed.

### Preparation Example 2

Preparation of α-amylase/anti-HbA1CFab′

(A) Preparation of GMB-Modified Amylase:

A maleimido group- was introduced into oc-amylase in the manner mentioned below. 100 μL of a solution (in DMF, 10 mg/mL) of GMBS (N-(γ-maleimidobutyryloxy)succinimide, by Dojin Chemical) was added to 1 mL of a solution of Bacillus subtilis α-amylase (5 mg/mL, 0.1 mol/L glycerophosphate buffer, pH 7.0), and reacted at room temperature for 1 hour. The reaction solution was subjected to gel permeation through a column of Sephadex G-25, and eluted with 0.1 mol/L glycerophosphate buffer (pH 7.0). The fraction having passed through the column was collected, and N-(γ-maleimidobutyryloxy)amidated amylase (GMB-modified amylase) was thus obtained. The concentration of the GMB-modified amylase solution obtained herein was 1.35 mg/mL.

(B) Preparation of Anti-Human HbA1C monoclonal antibody:

A monoclonal antibody IgG to human HbA1C was obtained in an ordinary manner. Briefly, immunized cells (spleen cells) obtained from an immunized mouse were fused with mouse myeloma cells, and cloned, and the intended antibody was collected from the clones. Concretely, 7 μg of natural human hemoglobin A1C dissolved in 1 mmol/L KCN (pH 7.45) was mixed with 143 μL of RPMI-1640 medium (containing 1 g/L sodium carbonate, 600 mg/L L-glutamine, 10 mmol/L HEPES; pH 6.8) and 200 μL of Freund's complete adjuvant, and the resulting mixture was subcutaneously injected to a mouse for immunizing it. Every two weeks, additional immunization was applied to the mouse. Finally, B lymphocytes were collected from the mouse spleen, and these were fused with mouse myeloma cells and cloned. From the resulting clones, selected was a cell strain capable of producing an antibody that specifically reacts with human HbA1C but substantially does not interact with any other hemoglobin subclass. The thus-selected antibody-producing cells were incubated, and a monoclonal specific antibody to human HbA1C, or that is, an anti-human HbA1C/mouse IgG was obtained through antibody purification.

(C) Preparation of Anti-Human HbA1C/IgGFab′:

20 mg of the thus-obtained anti-human HbA1C mouse IgG was dissolved in 10 mL of 0.1 mol/L acetate buffer (pH 5.5), and 500 μg of activated papain was added to it and stirred at 37πC. for 2 hours. The reaction solution was applied to a Superdex-200 gel column previously equilibrated with a 0.1 mol/L phosphate buffer (pH 6.0, containing 1 mmol/L EDTA), and eluted with the same phosphate buffer. The peak moiety eluted at around a molecular weight of 100,000 was collected, and an anti-human HbA1C/mouse IgGF(ab′)2 was thus obtained. 200 μL of an aqueous solution of 2-mercaptoethylamine hydrochloride (10 mg/mL) was added to 2 mL of the 0.1 mol/L phosphate buffer (pH 6.0) containing 10 mg of the anti-human HbA1C/IgGF(ab′)2, and stirred at 37° C. for 90 minutes. The reaction solution was subjected to gel permeation through a Sephadex G-25 gel column that had been previously equilibrated with a 0.1 mol/L phosphate buffer (pH 6.0), and the fraction having passed through the column was collected. An anti-human HbA1C/IgGFab′(hereinafter simply referred to as Fab′) was thus obtained.

(D) Preparation of α-amylase/Fab′ Bound Substance:

2 mg of the GMB-modified amylase prepared in (A) was added to 6.5 mL of the solution of anti-HbA1C/IgGFab′ (Fab′) (1.5 mg/mL) obtained in the above (C), and reacted overnight at 4° C. The resulting reaction solution was subjected to gel permeation through a Superdex-200 column previously equilibrated with 20 mmol/L glycerophosphate (pH 7.0, containing 10 mmol/L CaCl₂), and a fraction having a molecular weight of at least 300,000 was collected. The intended enzyme-labeled antibody (α-amylase/Fab′ bound substance) was thus obtained.

### Production Example 3

Construction of Dry Assay Element for HbA1C Quantitative Analysis

A crosslinking agent-containing reagent solution was applied onto a colorless transparent polyethylene terephthalate (PET) sheet (support) having a thickness of 180 μm and coated with a gelatin subbing layer, in such a manner that the coating amount of the constitutive components of the layer could be as follows. After dried, a reagent layer was formed on the support.

(Crosslinking agent-containing reagent solution)

| | | |
|---|---|---|
| Alkali-processed gelatin | 14.5 | g/m² |
| Nonylphenoxy-polyethoxyethanol | 0.2 | g/m² |
| (containing 9 or 10 oxyethylene units on average) | | |
| Glucose oxidase | 500 | IU/m² |
| Peroxidase | 15000 | IU/m² |
| Glucoamylase | 500 | IU/m² |
| 2-(4-Hydroxy-3,5-dimethoxyphenyl-4-[4- | 0.38 | g/m² |
| (dimethylamino)phenyl]-5-phenethylimidazole(leuco | | |
| dye)acetate | | |
| Bis[(vinylsulfonylmethylcarbonyl)amino]methane | 0.1 | g/m² |

An aqueous solution for adhesive layer was applied onto the reagent layer in such a manner that the coating amount of the constitutive components could be as follows. After dried, an adhesive layer was thus formed.

(Aqueous solution for adhesive layer)

| | | |
|---|---|---|
| Alkali-processed gelatin | 14.5 | g/m² |
| Nonylphenoxy-polyethoxyethanol | 0.2 | g/m² |
| (containing 9 or 10 oxyethylene units on average) | | |

Next, an aqueous solution containing reagents mentioned below was applied onto the surface of the adhesive layer in such a manner that the coating amount of the constitutive components could be as follows. Thus, the gelatin layer was swollen, and a tricot woven fabric produced by 36-gauge weaving of 50-denier PET spun yarn and having a thickness of about 250 μm was laminated on it by applying a light and uniform pressure to it. Thus, a porous spreading layer (woven fabric layer) was formed.

(Aqueous solution containing reagent)

| | |
|---|---|
| Nonylphenoxy-polyethoxyethanol | 0.2 g/m² |
| (containing 9 or 10 oxyethylene units on average) | |
| Bis[(vinylsulfonylmethylcarbonyl)amino]methane | 0.1 g/m² |

Next, a substrate-containing aqueous solution was applied onto the porous spreading layer in such a manner that the coating amount of the constitutive components could be as follows, and this was dried. Thus processed, the porous spreading layer (woven fabric layer) serves as a substrate layer and spreading layer.

(Substrate-containing aqueous solution)

| | | |
|---|---|---|
| Megafac F142D (by Dai-Nippon Ink) | 0.1 | g/m² |
| (fluorine-containing surfactant) | | |
| (containing 10 oxyethylene units on average) | | |
| Carboxymethylated starch | 5 | g/m² |
| Mannitol | 2 | g/m² |
| Amylase inhibitor | 1,000,000 | U/m² |
| (Fuji Rebio's amylase inhibitor “I-1001C”: | | |
| JP-A 61-74587) | | |

Next, an ethanol solution of the enzyme-labeled antibody (α-amylase/Fab′ bound substance) that had been prepared in Preparation Example 2 was applied to the substrate/spreading layer in a coating amount of 3 mg/m², infiltrated into it and dried. A multilayer dry assay element (1) for HbA1C assay was thus constructed.

The amylase inhibitor “I-1001C” used herein is an inhibitor to the same type of amylase that may be in a specimen, but this does not inhibit the enzymatic activity of the Bacillus subtilis α-amylase used herein as the labeling enzyme.

### Example 1

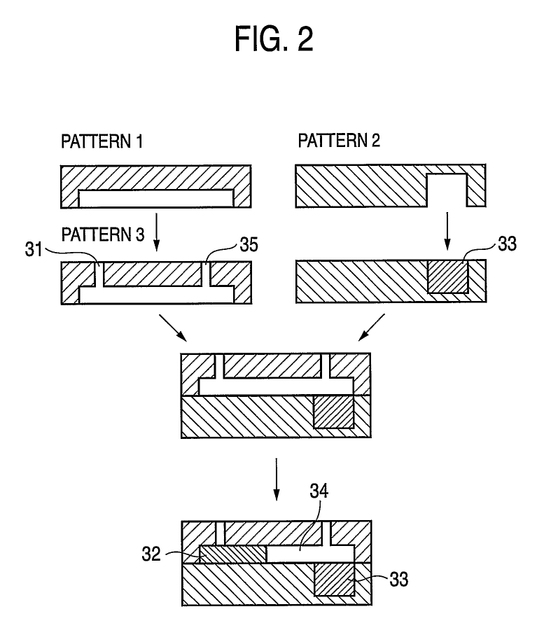

Construction of Integrated Assay Chip for Quantitative Determination of HbA1C (FIG. 2)

The dry assay element for HbA1C determination that had been produced in Production Example 2 was cut with 5 mm×5 mm, and fitted into the PDMS depression pattern (2) produced in Production Example 1. The PDMS depression pattern (2) was stuck to the PDMS depression pattern (3) (with an inlet for introducing) to construct the outline frame of an integrated assay chip.

Next, 5 μL of a pretreatment solution (10 mmol/L tris-HC1 buffer containing 0.1 v/v % Triton X-100 and 2 v/v % 2-butanol; pH 7.5) was gently introduced into it through the inlet for introducing 31, whereby an integrated assay chip 1 was thus completed. The flow channel that connects the pretreatment element and the assay element is fine and hydrophobic, and the hemolyzing reagent does not enter the assay element. (See FIG. 2.)

Capability Evaluation Test 1: Determination of HbA1C with Integrated Assay Chip 1 (Determination Example of the Invention)

(1) Preparation of Calibration Curve:

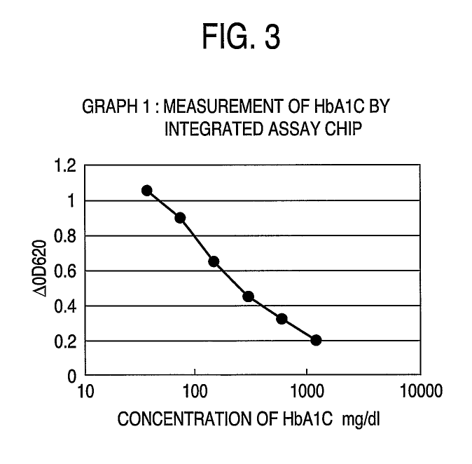

An HbA1C solution (13 mg/niL PBS, by Exocell) was diluted with 20 mmol/L Tris-HC1 (pH 7.5) buffer at intervals of 2ⁿ times (n=1, 2, 3 . . . ) to give 2ⁿ-diluted solutions having an HbA1C concentration of from 1200 mg/dL to 75 mg/dL. 0.2 μL of the diluted solution was forcedly injected into the integrated assay chip 1 through the inlet for introducing 31, and the solution in the pretreatment element was thus applied onto the assay element. Kept at 37° C., the reflection density at 650 nm of the specimen was measured with a spectral radiation luminance meter MCPD-2000 (by Otsuka Electronics). The difference between the reflection density OD after 2 minutes and that after 10 minutes (ΔOD2-10) was obtained, and a calibration curve was prepared from the data (FIG. 3).

(2) Determination of HbA1C in Whole Blood:

0.2 μL of a human whole blood specimen was forcedly injected into the integrated assay chip 1 through the inlet for introducing 31, and the hemolyzed solution in the pretreatment element was fed onto the assay element. Kept at 37° C., the reflection density at 650 nm of the specimen was measured with a spectral radiation luminance meter MCPD-2000 (by Otsuka Electronics). The difference between the reflection density OD after 2 minutes and that after 10 minutes (ΔOD2-10) was obtained, and the concentration of HbA1C of the blood specimen was calculated from the calibration curve obtained in the above (1). It was 720 mg/dL. ps (3) The above experiment (2) was repeated 6 times, and its reproducibility was confirmed. As the result, CV (Coefficient of variance) was 3.5%. ps Capability Evaluation Test 2: Determination of HbA1C with Multilayer Dry Assay Element (Comparative Example) ps (1) Preparation of Calibration Curve:

The multilayer dry assay element 1 of Production Example 2 was cut into a chip having a size of 15 mm², and fitted in a slide frame as in JP-A 57-63452. This is a comparative specimen, HbA1C quantitative assay slide (comparative example slide 1). Like in (1) in Capability Evaluation Test 1, an HbA1C solution (13 mg/nL PBS, by Exocell) was diluted with 20 mmol/L Tris-HC1 (pH 7.5) buffer at intervals of 2ⁿ times (n=1, 2, 3 . . . ) to give 2n-diluted solutions having an HbA1C concentration of from 1200 mg/dL to 75 mg/dL. 0.4 82 L of the diluted solution was mixed with 10 82 L of 10 mmol/L Tris-HC1 buffer (pH 7.5, containing 0.1 v/v % Triton X-100 and 2 v/v % 2-butanol), and then all the resulting mixture was spotted on a comparative example slide 1. The difference between the reflection optical density OD in 2 minutes after the spotting and that in 10 minutes after the spotting (66 OD2-10) was obtained, and a calibration curve was prepared from the data. ps (2) Determination of HbA1C in Whole Blood:

0.4 μL of the same human whole blood specimen as that used in Capability Evaluation Test 1 (specimen 1—this is the same as the specimen in Capability Evaluation Test 1) was mixed with 10 μL of 10 mmol/L Tris-HC1 buffer (pH 7.5, containing 0.1 v/v % Triton X-100 and 2 v/v % 2-butanol), and then all the resulting mixture was spotted on a comparative example slide 1. The difference between the reflection optical density OD in 2 minutes after the spotting and that in 10 minutes after the spotting (ΔOD2-10) was obtained, and the concentration of HbA1C of the blood specimen was calculated from the calibration curve obtained in the above (1). It was 715 mg/dL.

(3) The above experiment (2) was repeated 6 times, and its reproducibility was confirmed. CV was 4.5%.

(Comparison Between Capability Evaluation Tests 1 and 2)

As is obvious from the result in the above-mentioned Capability Evaluation Test 1, the integrated assay chip of the invention enables determination of HbA1C not requiring any substantial pretreatment of the specimen. Also as is obvious from the comparison between the results of Capability Evaluation Tests 1 and 2, the reproducibility of the integrated assay chip of the invention is good. The test data confirm the superiority of the capability of the integrated assay chip of the invention.

### [Apparatus Example 1]

Constitution of Measuring Apparatus

A photometric system of optical arrangement shown in FIG. 6 was prepared. Specifically, the following are provided as respective members.

Optical system: an inverted optical microscope

The following two sets of magnification at a CCD light-receiving part were provided.

Magnification of 0.33:33 μm/pixel at CCD part
Magnification of 1:10 μm/pixel at CCD part
Light source 12: Luminer Ace LA-150UX manufactured by Hayashi Watch-Works Co., Ltd.
Interference filter 14: transformed into monochrome each at 625 nm, 540 nm, or 505 nm
Dimmer filter 13: a glass filter ND-25 manufactured by HOYA Corporation and an own-made filter obtained by holing a stainless steel plate
CCD 16: an 8-bit black and white camera module XC-7500 manufactured by SONY Corporation
Data processor (Image processor): Image processing apparatus LUZEX-SE manufactured by Nireco Corporation

Means for correcting reflective optical density: The following 6 kinds of standard density panels (ceramic specification) manufactured by Fuji Photo Equipment Co., Ltd. were provided.

Standard density panels: A00
(reflective optical density: up to 0.05)

A05 (the same: 0.5)
A10 (the same: 1.0)
A15 (the same: 1.5)
A20 (the same: 2.0)
A30 (the same: 3.0)

### Example 2

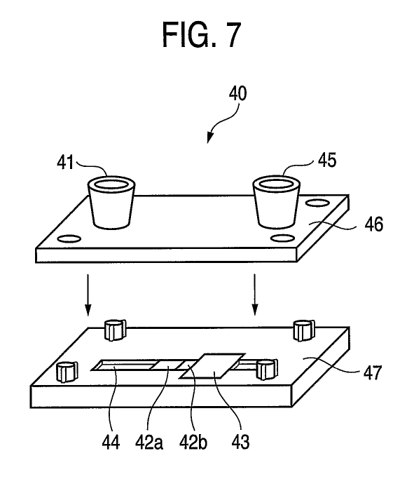

A microchip 40 of about 24 mm×28 mm size made of polystyrene resin (PS) shown in FIG. 7 was prepared. A glass fiber filter paper 42a for trapping erythrocytes and extracting plasma (manufactured by Whatman; GF/D) and a polysulfone porous membrane 42b (PSF manufactured by Fuji Photo Film Co., Ltd.) were mounted on a flow channel 44 having 2 mm width, 10 mm length, and 2 mm depth on a lower member 47 of the microchip so that the polysulfone porous membrane was placed toward the side of a multilayer dry assay element 43. The multilayer dry assay element 43 has a size in the arranged part of 5 mm width, 5 mm length, and 2 mm depth. FUJI DRI-CHEM Mount Slides GLU-P and TBIL-P (manufactured by Fuji Photo Film Co., Ltd.) as the multilayer dry assay elements 43 were each cut into a size of 2 mm width and 4 mm length and mounted so that GLU-P was placed in an upper position and TBIL-P in a lower position, and then the lower member 47 and the upper member 46 were adhered with a double-sided tape to maintain air tight and water tight.

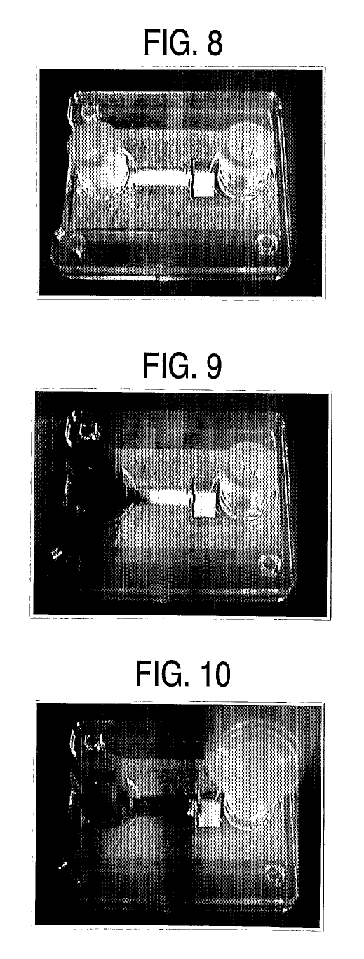

Subsequently, 100 μL of plain-specimend whole blood was inserted into a pipe 41 at the glass fiber filter paper 42a side of the upper member. After the whole blood was developed onto the glass fiber filter paper upon 10 to 20 seconds of standing, a Termo-syringe was mounted to a pipe 45 at a side opposite to the glass fiber filter paper side of the upper member and slight sucking was conducted. Extracted plasma by filtration leaked out of the polysulfone porous membrane 42b and was dropped onto the DRI-CHEM Mount Slide to gradually initiate coloring of DRI-CHEM Mount Slides GLU-P and TBIL-P (hereinafter also referred to as GLU-P and TBEL-P slides) (FIGS. 8 to 10). The time required from the injection of the plain-specimend whole blood to the dropping of the plasma onto the Mount Slides through the extraction of the plasma was 30 seconds.

Thus, the assay chip of the invention was small and enabled convenient and rapid assay on two or more assay items with a minute amount of whole blood. In this case, dry chemistry reagents for two items were employed as multilayer dry assay elements but the number of the items can be increased. Moreover, the assay was achieved based on coloring. Then, assay by photometry followed.

Coloring of the GLU-P and TBIL-P slides was photographed by the CCD camera using the optical system of [Apparatus Example 1] at the same time and the resulting images were processed using LUZEX-SE. An average quantity of received light at the center of each of the GLU-P and TBIL-P slides was determined and converted into optical density, whereby concentration of each of glucose and total bilirubin in the analyte was determined. In order to confirm correctness of the results measured using the CCD camera, concentration of each of glucose and total bilirubin in the analyte was determined using an automatic clinical test apparatus 7170 manufactured by Hitachi Ltd. The results are shown in Table 1. At this time, since measuring wavelengths were different from each other for the GLU-P and TBIL-P slides, photometry was conducted with sequentially changing the wavelength of the interference filter at intervals of 5 seconds as shown in Table 2. Thus, the assay chip of the invention enabled assay by photometry.

TABLE 1
Table 1: Values of components of whole
blood determined by CCD detection

| | Value determined by CCD | Value measured on Hitachi |
|---|---|---|
| | detection [mg/dL] | 7170 [mg/dL] |
| Glucose | 95 | 99 |
| Total bilirubin | 0.48 | 0.44 |

TABLE 2
Table 2: Irradiation sequence with sequentially
changing wavelength and light quantity

| Order | Wavelength [nm] |
|---|---|
| 1 | 505 |
| 2 | 540 |

* Order was sequentially changed in a manner of 1 → 2 → 1 → 2 → 1.

### INDUSTRIAL APPLICABILITY

The assay chip of the invention makes it possible to rapidly and accurately assay minor mount of components in a specimen in a simplified manner without substantially pretreating the specimen. The invention provides the assay chip not requiring any complicated pretreatment operation.

1. An integrated assay chip for assaying one or more components of a specimen, comprising: (1) one or more pretreatment elements for pretreating a specimen; (2) one or more multilayer dry assay elements capable of assaying one or more components of the pretreated specimen; and (3) one or more flow channels connecting the one or more pretreatment elements and the one or more multilayer dry assay elements.

2. An integrated assay chip for assaying one or more components of a specimen, comprising: (1) one or more pretreatment elements for pretreating a specimen; and (2) one or more multilayer dry assay elements capable of assaying one or more components of the pretreated specimen, wherein the one or more pretreatment elements and the one or more multilayer dry assay elements are connected inside the integrated assay chip.

3. The integrated assay chip according to claim 1 or 2, wherein the pretreatment is at least one selecting from the group consisting of a blood cell separation, hemolysis, dilution of specimen, degradation of protein, denaturation of protein, removal of endogenous substance and antigen-antibody reaction.

4. The integrated assay chip according to claim 1 or 2, wherein the one or more pretreatment elements (1) include an element for a blood cell separation, which contains at least one of a porous material and a water-insoluble substance that has an equivalent circle diameter of not more than 5 μm and a length equal to or longer than an equivalent circle diameter.

5. The integrated assay chip according to claim 1 or 2, wherein the one or more pretreatment elements (1) include an element for a blood cell separation, which contains any one of a glass fiber and a glass fiber filter paper.

6. The integrated assay chip according to claim 1 or 2, wherein the one or more pretreatment elements (1) include an element for a blood cell separation, which includes a blood filtration unit containing a glass fiber filter paper and a microporous membrane.

7. The integrated assay chip according to claim 4, wherein the element for a blood cell separation is an element utilizing a centrifugal force.

8. The integrated assay chip according to claim 1, wherein the one or more flow channels includes a micro flow channel having an equivalent diameter of 3 mm or less.

9. The integrated assay chip according to claim 1 or 2, which comprises a cartridge, in which the cartridge contains three elements of the one or more pretreatment elements, the one or more multilayer dry assay elements and the one or more flow channels, or two elements of one or more pretreatment elements and the one or more multilayer dry assay elements.

10. The integrated assay chip according to claim 1 or 2, wherein at least one of the one or more pretreatment elements and the one or more flow channels is formed on a substrate wide thereof by a micro-fabrication technique, and the one or more multilayer dry assay elements are bonded to the one or more flow channel.

11. A specimen assay method, comprising: applying a specimen to the integrated assay chip of claim 1 or 2; and leading the specimen through the one or more pretreatment elements and the one or more multilayer dry assay elements in this order.
